(19) United States
(12) Patent Application Publication
(10) Pub. No.: US 2024/0057589 A1
(43) Pub. Date: Feb. 22, 2024

(54) EFFERVESCENT SOLID DOSAGE FORM COMPOSITIONS CONTAINING ENVIRONMENTALLY SAFER ANTI-MICROBIAL COMPONENTS

(71) Applicant: ChemLink Laboratories, LLC, Kennesaw, GA (US)

(72) Inventor: Ryan Giffin MOORE, Kennesaw, GA (US)

(21) Appl. No.: 18/266,984

(22) PCT Filed: Dec. 21, 2021
(86) PCT No.: PCT/US2021/064526

§ 371 Date: Jun. 13, 2023

Related U.S. Application Data
(60) Provisional application No. 63/131,605, filed on Dec. 29, 2020.

(51) Int. Cl. A01N 25/12; A01N 25/30; A01N 25/26

(52) U.S. Cl. CPC A01N 25/12; A01N 25/30; A01N 25/26

(57) ABSTRACT

Provided are effervescent solid dosage form compositions that produce solutions when dissolved in a solvent that have increased shelf life and storage stability. The effervescent solid dosage form compositions include a sugar acid lactone in an amount in the range of about 1 wt % to about 15 wt %, based on the total weight of the composition; a preservative comprising salt of benzoic acid or sorbic acid or a combination thereof in an amount in the range of about 1 wt % to about 15 wt %, based on the total weight of the composition, and an effervescence generator.

### RELATED APPLICATIONS

Benefit of priority is claimed to U.S. Provisional Application No. 63/131,605, titled “EFFERVESCENT SOLID DOSAGE FORM COMPOSITIONS CONTAINING ENVIRONMENTALLY SAFER ANTI-MICROBIAL COMPONENTS,” filed Dec. 29, 2020, the subject matter of which is incorporated by reference herein in its entirety, where permitted.

### FIELD

This invention relates generally to effervescent solid dosage form compositions containing a salt of benzoic acid or sorbic acid in combination with a sugar acid lactone, such as gluconolactone, and methods for disinfecting or sterilizing a hard surface by application of solution containing one or more dissolved effervescent solid dosage form cleaner or sanitizer compositions provided herein. In particular, the solid dosage form compositions provided herein can be prepared as effervescent tablets, that when dissolved in a solvent, such as water, produce an environmentally friendly composition that can include a stable solution containing an anti-microbially effective amount of a benzoic acid or salt thereof or sorbic acid or salt thereof.

### BACKGROUND

Disinfecting, cleaning, and/or sanitizing of surfaces, in the home as well as in commercial and industrial settings, are necessary practices in order to promote and maintain good health. This is particularly true for the beverage, dairy, and food industries, in food packaging and preparation environments, and in service businesses. Failure to clean, disinfect, and/or sanitize equipment and other surfaces of contaminants can result in the growth of pathogenic microorganisms, yeasts, or molds. Routine cleaning, disinfection and/or sanitation is necessary to reduce microbial, yeast, or mold populations in order to protect consumers from potential health hazards associated with pathogenic microorganisms or toxins produced by such microorganisms, yeasts or molds.

Many existing antimicrobial compositions have drawbacks or undesirable properties that limit the ease of manufacture and/or use of the compositions. These include issues with availability and toxicity of ingredients, ease of use, efficacy, or having negative impacts on the items being treated, the individuals using the compositions, or on the environment. Examples of purported negative attributes or disadvantages of commonly used antimicrobial compositions are described in U.S. Pat. Appl. Publ. No. US20090074881 (Kielbania, Jr., 2009). New antimicrobial compositions should comply with the increasing demand for safer, more environmentally friendly compositions.

Effervescent tablets as consumer products have been around for almost one-hundred years. The two most well-known examples are Alka-Seltzer® heartburn relief formulations and Polident® denture cleaner formulations. Both of these products date back to the early 1930s and both use the combination of a carbonate and citric acid. However, both of these products produce solutions that are used completely by the consumer by the time the effervescence is completed or within a twenty-four hour period. Even with effervescent detergent products made in the mid-twentieth century these products were used up during the cleaning cycle. Therefore the need for a preservative to keep these solutions stable and not support microbial growth did not exist.

Only recently did the need for preservation systems to be built in effervescent tablet systems become necessary due to more effervescent tablet products being developed for the consumer. The reason that these effervescent tablet products will need preservation is due to the fact that when dissolved in water, the resulting solutions can be used over a course of weeks or months. These types of products will all require a preservation system to keep microbes, yeast or mold from growing in the prepared solutions before they are fully used by the consumer. Examples of these effervescent tablet products include multi-surface cleaners, glass cleaners, and foaming hand soap formulations.

Accordingly, there is a continuing need for compositions containing antimicrobial and biocidal compositions that exhibit improved stability, effective antimicrobial and biocidal activity, and that also are eco-friendly or “green.” The specific requirements for such compositions vary according to the intended application (e.g., disinfecting, sanitizing, sterilizing, etc.) and the governmental public health requirements associated with the intended application. Compositions having greater stability and antimicrobial and biocidal activity while being environmentally friendly could help meet a substantial public health need, and one that is not adequately addressed by current commonly-used antimicrobial compositions.

### SUMMARY

Provided are solid dosage form cleaner or sanitizer compositions containing a benzoic acid or salt thereof or sorbic acid or salt thereof in combination with a sugar acid lactone, such as gluconolactone, and an effervescence generator, in a solid dosage form, such as a tablet form, that when dissolved in a solvent produce a solution contain an antimicrobially effective amount of benzoic acid or salt thereof or sorbic acid or salt thereof, and that exhibit improved stability, enhanced antimicrobial efficacy, and that also are eco-friendly.

Provided are effervescent solid dosage form compositions that include a sugar acid lactone in an amount in the range of about 1 wt % to about 15 wt %, based on the total weight of the composition; a preservative comprising salt of benzoic acid or sorbic acid or a combination thereof in an amount in the range of about 1 wt % to about 15 wt %, based on the total weight of the composition; and an effervescence generator. The effervescence generator can include an acid in amount from about 20 wt % to about 60 wt % based on the total weight of the composition, and a base in an amount in the range of about 25 wt % to about 50 wt %, based on the total weight of the composition. The effervescence generator can include an acid in amount greater than 35 wt % but less than 60 wt % based on the total weight of the composition, and a base in an amount in the range of about 10 wt % to about 30 wt %, based on the total weight of the composition.

The effervescent solid dosage form compositions can be provide in the form of an agglomerate, granule, tablet, pellet, puck, brick, briquette, block, layered compression, or composite. In some applications, the effervescent solid dosage form compositions are provided as compressed tablets.

In the effervescent solid dosage form compositions provided herein, the sugar acid lactone can be selected from the group consisting of allohepturonolactone, allonolactone, alluronolactone, altrohepturonolactone, altronolactone, altruronolactone, arabinolactone, arabinuronolactone, galactohepturonolactone, galactonolactone, galacturonolactone, glucohepturonolactone, gluconolactone, glucuronolactone, gulohepturonolactone, gulonolactone, guluronolactone, idohepturonolactone, idonolactone, iduronolactone, lyxuronolactone, mannohepturonolactone, mannonolactone, mannuronolactone, ribonolactone, riburonolactone, talohepturonolactone, talonolactone, taluronolactone, xylonolactone and xyluronolactone and a combination thereof. The sugar acid lactone can be selected from the group consisting of a gluconolactone, a galactonolactone, a mannonolactone, a gulonolactone and a heptagluconolactone. The sugar acid lactone can be glucono-delta-lactone.

In the effervescent solid dosage form compositions provided herein, the acid of the effervescence generator can be a carboxylic acid. The acid can be a straight chain aliphatic carboxylic acid or a branched chain aliphatic carboxylic acid. The acid can be selected from the group consisting of acetic acid, adipic acid, citric acid, formic acid, fumaric acid, gluconic acid, glycolic acid, lactic acid, maleic acid, malic acid, oxalic acid, succinic acid, glutaric acid, malonic acid, and tartaric acid.

In the effervescent solid dosage form compositions provided herein, the base of the effervescence generator can be any basic material that is capable of generating a gas. The gas can be carbon dioxide. The base can be or include sodium bicarbonate, sodium carbonate, sodium sesquicarbonate, potassium carbonate, potassium bicarbonate, calcium carbonate, magnesium carbonate, magnesium oxide, sodium glycine carbonate, or a combination thereof.

In the effervescent solid dosage form compositions provided herein, the preservative can include any source of benzoic acid or sorbic acid. The preservative can be (a) sodium benzoate, potassium benzoate, calcium benzoate, or a combination thereof; or (b) sodium sorbate, potassium sorbate, calcium sorbate, or any combination thereof; or (c) a combination of any one or more of (a) and any one or more of (b).

The effervescent solid dosage form compositions provided herein can include a protective layer. The protective layer can include an acrylic polymer, a sugar, a starch, a maltodextrin, a polyethylene glycol, a film forming water soluble polymer, or a combination thereof. The protective layer can include a coating material selected from among adipic acid, azelaic acid, glutaric acid, malonic acid, oxalic acid, pimelic acid, sebacic acid, suberic acid, succinic acid, undecanedioic acid, dodecanedioic acid, tridecanedioic acid, hydroxypropyl cellulose, hydroxypropyl methylcellulose, polyvinyl acetate, hydroxyethyl cellulose, methylhydroxyethyl cellulose, methyl cellulose, ethyl cellulose, cellulose acetate, sodium carboxymethyl cellulose, polymers and copolymers of acrylic acid and methacrylic acid and esters thereof, starch, modified starch, maltodextrin, a wax, gum arabic, shellac, water soluble polyvinyl alcohol, a polyalkylene glycol, sodium polyacrylate, polyvinylpyrrolidone, and combinations thereof.

The effervescent solid dosage form compositions provided herein can include an additional component that does not negatively impact the activity of any of the component of the effervescent solid dosage form composition, such as the effervescence generator or preservative. The additional component can be selected from among an organic solvent, a surfactant, a foaming agent, a buffering salt, a tablet lubricant, a fragrance, a colorant, a chelant, an enzyme, an acid, a carbonate, a bicarbonate, a percarbonate, an acetate, a phosphate, sodium bisulfate, a wetting agent, a dispersing agent, a hydrotrope, a polyalkylene glycol, a polyglycol, a methoxypolyalkylene glycol, a polyglycol copolymer, a hexitol, a siloxane, a polysilane, a polysiloxane, sodium gluconate, a rheology control agent, a foam suppressant, a metal protectant, a corrosion inhibitor, a hydrogen peroxide generator in combination with a peracid catalyst, a bleaching agent, a bleach activator, an optical brightener, an anti-redeposition agent, and a combination thereof. When present in the composition, the surfactant can be selected from among a cationic surfactant, an anionic surfactant, a non-ionic surfactant, a zwitterionic surfactant, a silicone surfactant, a biosurfactant, and a combination thereof.

The effervescent solid dosage form compositions provided herein can include a humectant. The humectant can be selected from among glycerin, diglycerin, triacetin (glycerol triacetate), 1,3-butylene glycol, pentylene glycol, polyethylene glycol having a weight-average molecular weight of 800 or less, dipropylene glycol, panthenol, hyaluronic acid, sodium hyaluronate, and mixtures thereof. The humectant can be present in an amount of about 0.1 wt % to 10 wt % based on the weight of the solid dosage form.

The effervescent solid dosage form compositions provided herein can include an emollient. The emollient can selected from the group consisting of lanolin oil, cetyl alcohol, cetearyl alcohol, lauryl alcohol, oleyl alcohol, stearyl alcohol, PPG-15 stearyl ether, octyldodecanol, diisopropyl adipate, cetearyl octanoate. glycerin mono-, di-, and tri-esters, cholesterol, cholesterol esters, a linear silicone having a viscosity of from about 10 cP to about 100,000 cP at 25° C., a cyclomethicone, a light mineral oil, a petrolatum, a coconut oil, isopropyl myristate, soybean oil, stearic acid, caprylic acid, oleic acid, palmitic acid, lauric acid, octylpalmitate, octyl stearate, glyceryl stearate, and a combination thereof. The emollient can be present in an amount of about 0.1 wt % to 10 wt %, based on the weight of the solid dosage form.

The effervescent solid dosage form compositions provided herein can include a foam booster. The foam booster can be selected from among an aliphatic alcohol, an alkanolamide, and an amine oxide. The foam booster can be selected from among PEG-75, PEG-100, monoethanolamide, diethanolamide, N-methylethanolamide, glycerylmonocaprylate, cocamidopropyl hydroxysultaine, sorbitan sesquicaprylate, dodecyl alcohol, alkanolamide, monoethanolamide, diethanolamide, N-methyl-ethanolamide, lauramidopropyl betaine, and cocoamidopropyl betaine. The foam booster can be present in an amount of about 0.01 wt % to 15 wt %, based on the weight of the solid dosage form.

Also provided are solutions that contain the effervescent solid dosage form composition dissolved in a solvent. The solution can include from about 50 wt % to about 99.95 wt % of a solvent; and from about 0.05 wt % to about 50 wt % of the composition of claim 1 dissolved in the solvent. The solutions can be shelf stable and free of microbe growth. The solutions can have a pH from 4 to 6. The solutions can have a concentration of the preservative of the effervescent solid dosage form composition in an amount from 0.02 to 1 wt % based on the weight of the solution.

Also provided are methods of disinfecting a surface. The methods can include dissolving an effervescent solid dosage form composition provided herein in a solvent to form a solution; and applying the solution to the surface resulting in the destruction of, or prevention of the growth of, a microbe on the surface. The solvent used to dissolve the effervescent solid dosage form composition can include water, an alcohol, an aldehyde, a ketone, or a combination thereof. The disinfecting solution can be applied to the surface by spraying, wiping, immersion or direct application or any combination thereof. The disinfecting solution can be applied directly to a surface as a spray or fine mist. The disinfecting solution can be applied via a woven or nonwoven substrate, a brush, a sponge, a wipe or a cleaning pad, or any combination thereof.

Also provided are packaged systems that include an effervescent solid dosage form composition provided herein and a packaging material. The packaging material can be selected from the group consisting of glass, metal foil, treated metal foil, a metal foil pouch, plastic, plastic film, a plastic sheet, a blister pack, cardboard, a cardboard composite, paper and treated paper, and any combination thereof. The packaged system can include a container for dissolving the composition in a solvent, or a receptacle for containing or dispensing the dissolved composition, or both. The receptacle can be selected from the group consisting of a spray bottle, a sponge, a conventional hand sprayer container, an electric spray dispenser container, a bucket, a can, a drum, a towelette, a wipe, a pad, and any combination thereof.

Also provided are articles of manufacture. The articles of manufacture include an effervescent solid dosage form composition provided herein and (a) a container suitable for containing the effervescent solid dosage form composition; or (b) a set of instructions for preparing a cleaning solution or disinfectant solution by dissolving the effervescent solid dosage form composition in a solvent; or (c) a set of instructions for storing the effervescent solid dosage form composition; or (d) a material safety data sheet; or (e) a dispenser or applicator for a solution prepared by dissolution of the effervescent solid dosage form composition; or (f) any combination of two or more of (a), (b), (c), (d) and (e). The container can be made of or contain glass, acrylonitrile butadiene styrene (ABS), high impact polystyrene, polycarbonate, high density polyethylene, low density polyethylene, high density polypropylene, low density polypropylene, polyethylene terephthalate, polyethylene terephthalate glycol and polyvinylchloride and combinations thereof. The containers can include a barrier film to increase storage stability.

### DETAILED DESCRIPTION

### A. Definitions

Unless defined otherwise, all technical and scientific terms used herein have the same meaning as is commonly understood by one of skill in the art to which the inventions belong. All patents, patent applications, published applications and publications, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

As used herein, the singular forms “a,” “an” and “the” include plural referents unless the context clearly dictates otherwise.

As used herein, ranges and amounts can be expressed as “about” a particular value or range. “About” also includes the exact amount. Hence “about 5 percent” means “about 5 percent” and also “5 percent.” “About” means within typical experimental error for the application or purpose intended.

As used herein, “optional” or “optionally” means that the subsequently described event or circumstance does or does not occur, and that the description includes instances where the event or circumstance occurs and instances where it does not. For example, an optional component in a system means that the component may be present or may not be present in the system.

As used herein, “weight percent” or “wt %” refers to the concentration of a substance as the weight of that substance divided by the total weight of the composition and multiplied by 100.

As used herein, a “slow hydrolyzing acid” refers to an acid that slowly hydrolyzes with time to provide an additional carboxylic acid moiety, thereby further reducing the pH of a solution in which it is dissolved. Exemplary slow hydrolyzing acids are sugar acid lactones, which contain a lactone that hydrolyzes over time.

As used herein, “surfactant” refers to surface active molecules that absorb at the air/water, oil/water and/or oil/water interfaces, substantially reducing their surface energy. The term “detergent” is often used interchangeably with the term “surfactant.” Surfactants generally are classified depending on the charge of the surface active moiety, and can be categorized as cationic, anionic, nonionic and amphoteric surfactants.

As used herein, “effervescence generator” refers to a composition that gives off gas (e.g., carbon dioxide) bubbles when placed in an aqueous liquid.

As used herein, a “protective layer” refers to a layer that coats a surface in order to protect the surface from an influence of physical or chemical action applied from the surroundings.

As used herein, a “composite” refers to a mixture of two or more different ingredients in which the ingredients do not dissolve or merge completely, but which forms a substantially homogeneous material (i.e., a material without laminate structure or a composition gradient).

As used herein, a “layered compression” refers to composition containing two or more different ingredients in which the ingredients have a laminate structure or a composition gradient or both. The composition is formed by compressing the ingredients into a dry form, and can include multiple layers.

As used herein, an “agglomerate” refers to a material obtained by mixing two or more materials and agglomerating the resulting mixture. Such agglomeration is carried out using any of various known devices, examples of which include presses such as briquetting presses (e.g., cylinder briquette press, roller briquette press, ring roller briquette press), and also extruders and tumbling granulators (e.g., pan pelletizer, drum pelletizer). The shape of the agglomerate is not subject to any particular limitation. Any of various shapes may be used, such as blocky, granular, briquette-like, pellet-like or rodlike.

As used herein, “water soluble” refers to a compound that can be dissolved in water at a concentration of more than 1 wt %.

As used herein, “pouch” refers to a hollow sealable container.

As used herein, “water soluble or water dispersible protective pouch” refers to a pouch that at least partially dissolves in water or disperses in 1 liter of water at 21° C. within 10 minutes either with agitation or without agitation to allow for egress of the contents of the pouch into the surrounding water.

As used herein, a “stabilized benzoate or sorbate composition” has an enhanced stability, e.g., exhibits an increased shelf life or retains a higher level of effective amount of benzoic acid or sorbic acid over a given period of time because the benzoic acid or sorbic acid does not precipitate out of solution.

As used herein, “disinfect” refers to the process of destruction of, or prevention of the growth of, biological contaminants, which can include microbes.

As used herein, “disinfectant” refers to an agent that disinfects by destroying, neutralizing, or inhibiting the growth of biological contaminants, which can include microbes.

As used herein, “sanitary” means of or relating to the restoration or preservation of health, typically by removing, preventing or controlling an agent that may be injurious to health, such as microbes.

As used herein, “sanitize” means to make sanitary.

As used herein, “sanitizer” refers to an agent that sanitizes.

As used herein, “microbes” refers to any organism that is a member of the phylogenetic domains bacteria and archaea, as well as unicellular and filamentous fungi (such as yeasts and molds), unicellular and filamentous algae, unicellular and multicellular parasites, and viruses. Exemplary microbes include bacteria, e.g., Gram-positive and Gram-negative cocci, Gram-positive and Gram-negative straight, curved, helical or vibroid and branched rods, sheathed bacteria, sulfur-oxidizing bacteria, sulfur or sulfate-reducing bacteria; spirochetes; actinomycetes and related genera; myxobacteria; mycoplasmas; rickettsias; chlamydias; cyanobacteria; archaea; fungi; yeasts; parasites; viruses; and algae.

As used herein, “eco-friendly” means not harmful to, or having minimal negative impact on, the environment.

As used herein, a “solid” refers to a hardened composition that does not flow perceptibly and that substantially retains its shape under moderate stress, pressure or gravity.

As used herein, “antimicrobial activity” refers to partial or complete inhibition of growth of a microbe, or causing lysis of a microbe, or both. A sanitizer and a disinfectant are exemplary agents that have antimicrobial activity.

As used herein, “antimicrobial composition” is a composition that partially or completely inhibits growth of a microbe, or causes lysis of a microbe, or death of the microbe, or any combination thereof.

As used herein, “acidic range” means a pH less than 7.

As used herein, “metal protector” refers to a material, substance, composition, or compound that protects a metal from its environment.

As used herein, “corrosion inhibitor” refers to a material, substance, composition, or compound that reduces, decreases, diminishes, lowers, or minimizes the corrosion of a metal or metal alloy from the surface of a metal component in the presence of metal corroding agent.

As used herein, “room temperature” means an ambient temperature in the range of from about 20° C. to about 25° C.

As used herein, a “clear solution” refers to a clear or transparent solution that is devoid of any visual particles or precipitate when viewed with an unaided eye.

As used herein, “tf” means ton-force (metric), where 1 metric ton-force (tf) equals about 9.8 kilonewtons (kN) or 1000 kilogram-force (kg). A gauge of a tablet press reading 1 metric ton means that 1 metric-ton force (tf) is applied to compress the tablet.

As used herein, “humectant” refers to a water-attracting or moisture-attracting sub stance.

As used herein, an “emollient” refers to a material used to soften or soothe a surface. An emollient can prevent or relieve dryness, prevent or mitigate drying, reduce itchiness of dry skin, maintain moisture of skin, or prevent skin cracking.

As used herein, a “foam booster” refers to a substance that increases foam production or stabilizes lather or foam. Such a substance can also be called a “foam stabilizer”. Foam boosters can also improve the quality and texture of the foam. Foam boosters can make the foam thicker, creamier, or longer lasting.

As used herein, a “biosurfactant” is a surface-active agent of biological origin.

### B. Solid Dosage Compositions

Provided herein are effervescent solid dosage compositions that when dissolved in a solvent produce a cleaning or disinfecting solution that contains a stabilized benzoate or sorbate having antimicrobial activity.

Effervescent tablets offer many advantages in producing environmentally-friendly, sustainable, and safer products for the consumer. These advantages include: not using water during the production process, reducing pollution by not shipping or storing water during shipping, and using safer ingredients outlined by the US EPA Safer Chemical Ingredient List (SCIL). The two best preservative examples which work well in the formulation and production of effervescent tablet systems from the US EPA SCIL are sodium benzoate and potassium sorbate. Both of these safer preservative ingredients are listed as “100% Full Green Circle” which is the US EPA Safer Choice's highest rating for ingredient safety. These characteristics of sodium benzoate and potassium sorbate make these ingredients ideal for developing effervescent tablet products that the consumer will need to store as prepared solutions for weeks or months.

Another important factor in developing an effervescent product is the speed at which an effervescent tablet will dissolve. Tablets that take more than 45 minutes to 1 hour to fully dissolve are not usually desired by the consumer. Certainly, total tablet size and weight will make a difference in how fast a tablet will dissolve into water. Water temperature is also a factor in helping a tablet dissolve into water. However, most consumers across the globe will prefer to use “room-temperature” water (ambient), and in some geographical locations, access to hot water may be limited or hot water may not easily be available. To make a tablet dissolve faster than 45 to 60 minutes in ambient temperature water or even cold water usually takes an acid amount that is greater than 60% by weight of the tablet formulation.

Formulating effervescent tablet products with such high acid content, however, can lead to two problems. The first problem is that a formulation containing acid in an amount of 60% by weight or more of the tablet formulation allows only 40% or less by weight of other ingredients that can be included in the formulation, hampering formulation design and making it much more difficult to formulate an effective cleaner or sanitizer solution. The second problem is that this high amount of acid can cause the pH of the solution to drop below pH 4.5 before the rest of the components can buffer the solution to the desired pH. For example, the desired pH could be 5.5 for a foaming hand soap.

The reason this drop in pH is problematic is because when using either sodium benzoate or potassium sorbate to provide antimicrobial activity, these ingredients will turn into benzoic acid and sorbic acid, both of which are not readily soluble and can crystallize or precipitate out of solution at certain concentrations when the pH is below 4.5. When benzoic acid and sorbic acid have crystallized or precipitated out of solution, the crystallized or precipitated material is unsightly, can clog the dispensing pumps used for administering or applying the solution, and most importantly the precipitated or crystallized benzoic acid and sorbic acid are not in solution working as preservatives and antimicrobials, leaving the solutions vulnerable to microbial growth.

To solve this problem, provided herein are solid dosage form compositions that include an acid in amount that is above 35 wt % but less than 60 wt % to keep the solution from dropping in pH too fast and too low. The compositions include a sugar acid lactone, e.g., glucono-delta-lactone, as a pH modulator. The sugar acid lactone slowly forms an acid in solution. The amount of sugar acid lactone in the composition can be an amount in the range of about 1 wt % to about 15 wt %, based on the total weight of the composition. It has been found that this combination of ingredients inhibits the rapid drop in pH, allowing the sodium benzoate or potassium sorbate to remain in solution while being converted into an antimicrobially effective amount of benzoic acid or sorbic acid, respectively, but still allowing the effervescence generator to cause dissolution of the effervescent in less than 45 minutes when added to a solvent.

Many of the antimicrobial compositions in use can have a negative impact on the environment. For example, some halogenated phenolics can present environmental concerns because they are difficult to degrade. Some antimicrobial compounds, such as hypochlorite, can react with many organic materials, which can result in the production of carcinogenic compounds. Some surfactants used as antimicrobials can be toxic to aquatic life. Some antimicrobials in use are not biodegradable and can be persistent in the environment.

The compositions provided herein have antimicrobial properties and are “green” or eco-friendly. Eco-friendly compositions include those recognized to have minimal negative impact on the environment. For example, the United States Environmental Protection Agency's Design for the Environment Antimicrobial Pesticide program allows for special labeling of compositions that can meet certain requirements. These include that the product is unlikely to cause developmental, mutagenic, neurotoxic or reproductive harm, does not require the use of EPA-mandated personal protective equipment, and has no unreasonable or unresolved adverse effects reported. Such products must demonstrate antimicrobial activity on hard, non-porous surfaces.

The compositions and methods provided herein are environmentally safe. The compositions provided herein have superior or equal antimicrobial performance when compared to other standard antimicrobial agents, with the added benefits of being particularly “green” or eco-friendly compositions, and the ability to be formulated as a stable dry powder or tablet that can be reconstituted, which further decreases any negative environmental impact. The solid dosage form compositions provided herein, and the liquid solutions prepared by dissolving the solid dosage form compositions provided herein in a solvent, can be stored and maintain stability for a prolonged period of time, which is another advantageous outcome of the present technology.

The compositions provided herein address a market need for a solid dosage form cleaning and/or disinfecting composition containing an environmentally preferred antimicrobial that is stable in dry form and that yield an antimicrobially effective solution when dissolved in a solvent. The compositions provided herein stabilize a benzoate or sorbate antimicrobial so that the benzoate or sorbate does not precipitate as benzoic acid or sorbic acid, respectively, while allowing an antimicrobially effective amount of benzoic acid or sorbic acid to remain in solution.

The antimicrobial compositions provided herein can be maintained in their dry form until ready for use. The dry form, e.g., a solid dosage form that can be an agglomerate, granule, tablet, pellet, puck, brick, briquette, block, layered compression, or composite, can include a polymer coating as a protective layer. The polymer coating can be applied to the surface of the solid form of the cleaner or sanitizer composition. The protective layer can be in the form of a film, packet, pouch, sheath or envelope that surrounds, partially or completely, the solid form of the antimicrobial composition.

The effervescent solid dosage forms provided herein are substantially stable at room temperature for a year or more. When dissolved in a solvent, the effervescent solid dosage forms provided herein result in formation of a cleaning or disinfecting solution containing an antimicrobially effective amount of a benzoate or sorbate, and the solutions are shelf stable, no precipitates form over time, and the solutions can remain free of microbial growth for a year or more.

### C. Components of Eco-Friendly Effervescent Solid Dosage Forms

Provided herein are eco-friendly, environmentally acceptable effervescent solid dosage forms that include antimicrobial compositions as a preservative and that, when dissolved in a solvent, produce a solution containing an antimicrobially effective amount of a benzoate (salt of benzoic acid) or sorbate (salt of sorbic acid) as a preservative. The compositions stabilize the benzoate or sorbate in solution so that benzoic acid or sorbic acid does not precipitate out of solution, which would reduce the biocidal effectiveness. The effervescent solid dosage forms provided herein include a preservative, a sugar acid lactone, and an effervescence generator, and generally contain additional components to provide cleaning and/or sanitizing functionality, or organoleptic properties.

The effervescent solid dosage forms provided herein can be dissolved in any solvent. Exemplary solvents include water, an alcohol, an aldehyde, and a ketone and combinations thereof. For many applications, the solvent includes water.

1. Preservative

The effervescent solid dosage form compositions provided herein include a preservative that is an antimicrobial composition. The preservative can include a salt of benzoic acid or sorbic acid or any combination thereof. For example, the preservative can include sodium benzoate, potassium benzoate, calcium benzoate, sodium sorbate, potassium sorbate, calcium sorbate, or any combination thereof. Many of the salts of benzoic acid and sorbic acid can be used as a direct food-additive and in pharmaceutical and cosmetic products. The salts of benzoic acid and/or sorbic acid can be used for in-container control of bacteria, molds, and yeast in household and industrial products.

Among the salts of benzoic acid and sorbic acid, sodium benzoate and potassium sorbate can be preferred. Both of these salts are considered safer preservative ingredients and are listed as “100% Full Green Circle” which is the US EPA Safer Choice's highest rating for ingredient safety. These characteristics of sodium benzoate and potassium sorbate make these ingredients ideal for developing effervescent tablet products that when added to a solvent yield a solution that the consumer will need to store as prepared for weeks or months.

Bacterial and fungal growth can be inhibited in solutions by including a concentration of sodium benzoate or potassium sorbate in the range of 50 to 60,000 mg/L. In some formulations, concentrations of sodium benzoate or potassium sorbate of about 0.025% to 0.1% based on the total weight of the solution have been found to be generally sufficient to preserve a liquid product.

In the effervescent solid dosage forms provided herein, the preservative can be present in an amount of 1 wt % to 15 wt % based weight of the solid dosage form, or in an amount of about 5 wt % to about 10 wt % based weight of the solid dosage form. It is to be understood that all values and ranges between these values and ranges are encompassed by the compositions and methods provided herein. For example, the amount of preservative present in the effervescent solid dosage forms provided herein can be 1.0 wt %, 1.5 wt %, 2 wt %, 3.75 wt %, 4.25 wt %, 5 wt %, 6 wt %, 7 wt %, 7.5 wt %, 8 wt %, 8.5 wt %, 9 wt %, 9.5 wt %, 10 wt %, 10.5 wt % 11 wt %, 11.5 wt %, 12 wt %, 12.5 wt %, 13 wt %, 13.5 wt %, 14 wt %, 14.5 wt %, or 15 wt %.

2. Sugar Acid Lactone

The effervescent solid dosage form compositions provided herein include a sugar acid lactone. The sugar acid lactones are acids that slowly hydrolyze to provide an additional carboxylic acid moiety, thereby reducing the pH of a solution in which they are dissolved. Any sugar acid lactone can be used in the compositions provided herein. Exemplary sugar acid lactones include the alpha and beta forms of allohepturonolactone, allonolactone, alluronolactone, altrohepturonolactone, altronolactone, altruronolactone, arabinolactone, arabinuronolactone, galactohepturonolactone, galactonolactone, galacturonolactone, glucohepturonolactone, gluconolactone, glucuronolactone, gulohepturonolactone, gulonolactone, guluronolactone, idohepturono-lactone, idonolactone, iduronolactone, lyxuronolactone, mannohepturonolactone, mannonolactone, mannuronolactone, ribonolactone, riburonolactone, talohepturonolactone, talonolactone, taluronolactone, xylonolactone and xyluronolactone.

In some embodiments, the sugar acid lactone is selected from among the group consisting of a gluconolactone, a galactonolactone, a mannonolactone, a gulonolactone and a heptagluconolactone and combinations thereof. In some embodiments, the sugar acid lactone is selected from among D-glucono-delta-lactone [GDL, CAS-No. 90-80-2], D-galactono-gamma-lactone [CAS-No. 2782-07-2], L-mannono-gamma-lactone [CAS-No. 22430-23-5], D-gulono-gamma-lactone [CAS-No. 6322-07-2], L-gulono-gamma-lactone [CAS-No. 1128-23-0], α-D-heptaglucono-gamma-lactone [CAS-No. 60046-25-5] and combinations thereof. In some embodiments, the sugar acid lactone is D-glucono-[delta]-lactone [CAS-No. 90-80-2]. In some embodiments, the sugar acid lactone is a glucono-delta-lactone, D-glucofuranurono-6,3-lactone, or glucuronolactone or a combination thereof. In some embodiments, the sugar acid lactone is a gluconolactone, a galactonolactone, a mannonolactone, a gulonolactone, a glucono-delta-lactone or a heptagluconolactone or a combination thereof. In some embodiments, the sugar acid lactone is a glucono-delta-lactone.

The sugar acid lactones are commercially available or can be synthesized using known methods. For example, many alduronic acids form intramolecular lactones by removing one mole of water between the carboxyl group and one hydroxyl group. These internal esters of alduronic acids are spontaneously formed upon heating suitable alduronic acids in which formation of a 1-4 or gamma-lactone structure is possible, or in which formation of a 1-5 or delta-lactone structure is possible. The alduronic acids are a group of compounds which are obtainable by oxidation of the terminal alcohol group of aldoses. The penturonic acids can be converted to penturonolactones using similar methods.

The sugar acid lactone can be present in the solid form compositions provided herein in an amount from about 1 wt % to about 15 wt %, or from about 5 wt % to about 15 wt %, or from about 5 wt % to about 10 wt %, or from about 10 wt % to about 15 wt %, or from about 7.5 wt % to about 15 wt %, or from about 5 wt % to about 7.5 wt %, or from about 1 wt % to about 10 wt %, or from about 1 wt % to about 7.5 wt %, or from about 1 wt % to about 5 wt %, based on the total weight of the solid form composition. It is to be understood that all values and ranges between these values and ranges are encompassed by the compositions and methods provided herein. For example, the sugar acid lactone can be present in the solid form compositions provided herein in an amount of 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt % 11 wt %, 11.5 wt %, 12 wt %, 12.5 wt %, 13 wt %, 13.5 wt %, 14 wt %, 14.5 wt %, or 15 wt %.

In some applications, the amount of sugar acid lactone is selected so that the ratio of preservative to sugar acid lactone present in the solid dosage form can be 2:1 to 1:2. For example, the ratio of preservative to sugar acid lactone present in the solid dosage form can be 2:1, 1.75:1, 1.5:1, 1.25:1, 1:1, 1:1.25, 1:1.5, 1:1.75, or 1:2.

3. Effervescence Generator

The effervescent solid dosage form compositions provided herein is water soluble and rapidly disintegrates in a solvent, such as water. The effervescent solid dosage form compositions provided herein can dissolve in a solvent to form a clear solution. When placed in excess water at room temperature, the effervescent solid dosage form compositions provided herein can dissolve in less than 45 minutes, or less than 30 minutes, or less than 20 minutes; or less than 15 minutes, or less than 10 minutes, or less than 5 minutes. The uniformity and clarity of the resulting solution can be determined by viewing with the naked eye.

To formulate the effervescent solid dosage form compositions provided herein, an effervescence generator is included. In some embodiments, the effervescence generator includes a combination of an acid and a base. When contacted with a solvent that includes water, the effervescence generator is activated, liberating the acid and base, which react with each other to produce a gas, e.g., carbon dioxide gas.

Examples of useful acids that can be included in the effervescence generator are any of the carboxylic acids, sodium acid pyrophosphate, and mixtures thereof. Any carboxylic acid known in the art compatible with the other components of the composition can be used. The carboxylic acid can be a straight chain aliphatic carboxylic acid or a branched chain aliphatic carboxylic acid or a combination thereof. Exemplary carboxylic acids include acetic acid, adipic acid, ascorbic acid, citric acid, formic acid, fumaric acid, gluconic acid, glutaric acid, glycolic acid, lactic acid, sorbic acid, succinic acid and sulfamic acid and combinations thereof. In some embodiments, the carboxylic acid is selected from the group consisting of acetic acid, adipic acid, citric acid, formic acid, fumaric acid, gluconic acid, glycolic acid, lactic acid, maleic acid, malic acid, oxalic acid, succinic acid, glutaric acid, malonic acid, and tartaric acid. In some embodiments, the compositions can include citric acid, alone or in combination with another carboxylic acid. In some applications, the formulation can include a solid acetic acid as described in U.S. Pat. No. 8,859,482 (Moore, 2014).

The acid can be present in the effervescence generator in an amount of from 10 wt % to about 85 wt %, or from about 15 wt % to about 75 wt %, or from about 20 wt % to about 60 wt %, or from about 25 wt % to about 50 wt %, based on the weight of the effervescence generator. It is to be understood that all values and ranges between these values and ranges are encompassed by the compositions and methods provided herein.

The acid present in the effervescence generator can serve a dual function of generating bubbles when coming into contact with the base of the effervescence generator, as well as serving as a pH modulator to reduce the pH, participating as a functional buffer, acting as a chelating agent, improving the effectiveness of a surfactant acing as a detergent or cleansing agent, and depending on the acid selected, acting as an anti-oxidant.

The base of the effervescence generator is capable of generating a gas, such as carbon dioxide. Examples of suitable carbonate bases include sodium bicarbonate, sodium carbonate, sodium sesquicarbonate, potassium carbonate, potassium bicarbonate, calcium carbonate, magnesium carbonate, magnesium oxide, sodium glycine carbonate, and mixtures thereof. The effervescence generator can include a base in an amount of from about 10 wt % to about 85 wt %, or from about 15 wt % to about 75 wt %, or from about 20 wt % to about 60 wt %, or from about 25 wt % to about 50 wt %, based on the weight of the effervescence generator. In some embodiments, the effervescence generator can include an alkali metal carbonate and an acid. The alkali metal carbonate can be anhydrous potassium carbonate, hydrated potassium carbonate, anhydrous sodium carbonate, or hydrated sodium carbonate or a combination thereof.

The amount of effervescence generator included in the solid dosage form compositions provided herein can vary, such as in an amount from about 20 wt % to about 80 wt % by weight of the solid dosage form composition. It is to be understood that all values and ranges between these values and ranges are encompassed by the compositions and methods provided herein. For example, the effervescence generator can be included in the solid dosage form compositions provided herein in an amount of 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, or 80 wt %, based on the weight of the solid dosage form composition.

The effervescence generator can include an amount of acid relative to the base in excess of that needed to neutralize the base and form bubbles (e.g., carbon dioxide). The excess acid then can interact with the acid salt formed by the reaction of the acid with the base, thereby forming a buffer in situ. The buffer can have a targeted pH range by adjusting the amount of excess acid in the effervescence generator, or the acid included in the effervescence generator, or both. For example, by using an excess of citric acid, during dissolution of the tablet, the citric acid reacts with the base, such as sodium carbonate, to form bubbles by releasing carbon dioxide, resulting in the formation of sodium citrate, which can interact with the free citric acid to form a citrate buffer. Depending on the amount of free citric acid and sodium citrate in solution, the buffered solution can have a pH in the range of about 3.0 to 6.0. The amount of free acid can be controlled so that the pH does not result in the precipitation of the benzoate and/or sorbate preservative.

Similarly, by using an excess of acetic acid, during dissolution of the tablet, the acetic acid reacts with the base, such as sodium carbonate, to form bubbles by releasing carbon dioxide, resulting in the formation of sodium acetate, which can interact with the free acetic acid to form an acetate buffer. Depending on the amount of free acetic acid and sodium acetate in solution, the buffered solution can have a pH in the range of about 3.6 to 5.6. The amount of free acid can be controlled so that the pH does not result in the precipitation of the benzoate and/or sorbate preservative. Any one of acetic acid, adipic acid, ascorbic acid, citric acid, formic acid, fumaric acid, gluconic acid, glutaric acid, glycolic acid, lactic acid, sorbic acid, succinic acid, sulfamic acid, or combinations thereof, can be used to react with the base to form a buffer in situ.

4. Additional Components

The effervescent solid dosage forms provided herein can include one or more additional components. Exemplary additional components include, e.g., organic solvents, surfactants, foaming agents, a buffering salt, tablet lubricants, fragrances, colorants, chelants (e.g., EDTA, iminodisuccinic acid salts (available as Baypure ° CX 100 from Lanxess Deutschland GmbH, Leverkusen Germany) and methylglycine diacetic acid (Trilon® M from BASF, Florham Park, NJ)), enzymes, acids, carbonates or bicarbonates, percarbonates, acetates, phosphates, sodium bisulfate, wetting agents, dispersing agents, hydrotropes, polyalkylene glycols, polyglycols, methoxypolyalkylene glycols, polyglycol copolymers, hexitols, siloxanes, polysilanes, polysiloxanes, sodium gluconate, rheology control agents, foam suppressants, metal protectants, corrosion inhibitors, a hydrogen peroxide generator in combination with a peracid catalyst, bleaching agents, bleach activators, optical brighteners, anti-redeposition agents, and other functional additives, alone or in combination. In some applications, the formulation includes an expanded percarbonate as described in U.S. Pat. No. 8,652,434 (Moore et al., 2014).

The effervescent solid dosage forms provided herein can include a bleaching agent. Exemplary bleaching agents include bleach activators, hydrogen peroxide, sources of hydrogen peroxide, pre-formed peracids, alkali metal hypochlorites, monochloramine, dichloramine, alkali metal dichloroisocyanurates, chlorinated trisodium phosphate and mixtures thereof.

Exemplary bleach activators include an acylated alkylene diamine, benzoyl peroxide, benzoyl caprolactam, tetraacetyl glycoluril, N-acylated hydantoin, hydrazine, triazole, hydratriazine, urazole, di-ketopiperazine, sulfurylamide, 6-nonyl-amino-6-oxoperoxy-caproic acid, cyanurate, a carboxylic acid anhydride, decanoyl-oxybenzene sulphonate sodium-acetoxy-benzene sulfonate, sodium-benzoyloxy benzene sulfonate, sodium-lauroyloxy-benzene sulfonate, sodium-isononanoyloxy benzene sulfonate, acylated sugar derivatives, pentaglucose, nonanoyloxybenzene sulfonate, and combinations thereof.

The effervescent solid dosage forms provided herein can include a surfactant. Any surfactant known in the art can be used, including cationic, anionic, non-ionic and zwitterionic surfactants, including silicone surfactants, and biosurfactants. Exemplary non-ionic surfactants include Tergitol NP-9® (Dow Chemical Co., Midland, MI), a nonionic, nonylphenol ethoxylate surfactant, Tergitol NP-33 [9016-45-9] Synonyms: alpha(nonylphenyl)-omega-hydroxypoly(oxy-1,2-ethanediyl); antarox; nonylphenoxypoly(ethyleneoxy)ethanol; nonylphenyl polyethyleneglycol ether, nonionic; nonylphenyl polyethylene glycol ether; PEG-9 nonyl phenyl ether; POE (10) nonylphenol; POE (14) nonylphenol; POE (15) nonyl phenol; POE(15) Nonyl Phenyl Ether; POE(18) Nonyl Phenyl Ether; POE (20) nonylphenol; POE(20) Nonyl Phenyl Ether; POE (30) nonylphenol; POE (4) nonylphenol; POE (5) nonylphenol; POE (6) nonylphenol; POE (8) nonylphenol; polyethylene glycol 450 nonyl phenyl ether; polyethylene glycol 450 nonyl phenyl ether, nonionic surfactant; polyethylene glycols mono(nonylphenyl)ether; polyethylene mono(nonylphenyl)ether glycols; polyoxyethylene (10) nonylphenol; polyoxyethylene (14) nonylphenol; polyoxyethylene (1.5) nonyl phenol; polyoxyethylene (20) nonylphenol; polyoxyethylene (30) nonylphenol; polyoxyethylene (4) nonylphenol; polyoxyethylene (5) nonylphenol; polyoxyethylene (6) nonylphenol; polyoxyethylene (8) nonylphenol; Polyoxyethylene (9) Nonylphenyl Ether; polyoxyethylene(n)-nonylphenyl ether; Polyoxyethylene nonylphenol; POE nonylphenol; Protachem 630; Sterox; Surfionic N; T-DET-N; Tergitol NP; Tergitol NP-14; Tergitol NP-27; Tergitol NP-33; Tergitol NP-35; Tergitol NP-40; Tergitol NPX; Tergitol TP-9; Tergitol TP-9 (non-ionic); Triton N; Triton X; Dowfax 9N; ethoxylated nonylphenol; glycols, polyethylene, mono(nonylphenyl) ether; Igepal CO; Igepal CO-630; macrogol nonylphenyl ether; Makon; Neutronyx 600; Nonipol NO; nonoxinol; nonoxynol; Nonoxynol-15; Nonoxynol-18; Nonoxynol-20; nonyl phenol ethoxylate; nonylphenol polyethylene glycol ether; nonylphenol, polyoxyethylene ether; nonylphenoxypolyethoxyethanol; nonionic surfactants having a polyalkylene oxide polymer as a portion of the surfactant molecule, such as chlorine-, benzyl-, methyl-, ethyl-, propyl-, butyl- and other similar alkyl-capped polyethylene glycol ethers of fatty alcohols; polyalkylene oxide free non-ionics such as alkyl polyglycosides; sorbitan and sucrose esters and their ethoxylates; alkoxylated ethylene diamine; alcohol alkoxylates such as alcohol ethoxylate propoxylates, alcohol propoxylates, alcohol propoxylate ethoxylate propoxylates, alcohol ethoxylate butoxylates; nonylphenol ethoxylate, polyoxyethylene glycol ethers; carboxylic acid esters such as glycerol esters, polyoxyethylene esters, ethoxylated and glycol esters of fatty acids; carboxylic amides such as diethanolamine condensates, monoalkanolamine condensates, polyoxyethylene fatty acid amides; and polyalkylene oxide block copolymers including an ethylene oxide/propylene oxide block copolymer such as those commercially available under the trademark PLURONIC® (BASF-Wyandotte).

Silicone surfactants also can be used. For example, in some embodiments, the silicone surfactant includes a surface active polydiorganosiloxane, such as described in U.S. Pat. No. 4,421,656 (Donatelli et al., 1983). In some embodiments, the silicone surfactant can be selected from among dimethicone copolyols and alkyl dimethicone copolyols and blends thereof. Examples of such silicone surfactants include the blends of dimethicone copolyol and cyclomethicone, such as sold by Dow Corning (Midland, MI) under the name DC3225C or DC2-5225C, a polyalkyl polyether polysiloxane copolymer having an alkyl radical containing from 5 to 22 carbon atoms, such as cetyl dimethicone copolyol, such as that sold under the name Abil® EM-90 by Evonik Industries AG (Essen, Germany), the mixture of dimethicone copolyol and cyclopentasiloxane (85/15), such as that sold under the name Abil® EM-97 by Goldschmidt, linear-type polyether-modified silicone emulsifiers, including methyl ether dimethicones, such as PEG-3 methyl ether dimethicones, PEG-9 methyl ether dimethicones, PEG-10 methyl ether dimethicones, PEG-11 methyl ether dimethicones, and butyl ether dimethicones (available from Shin-Etsu (Akron, Ohio); branched-type polyether-modified silicone emulsifiers, such as PEG-9 polydimethylsiloxyetheyl dimethicone (Shin-Etsu), alkyl co-modified branched-type polyether silicones, such as lauryl PEG-9 polydimethylsiloxyethyl dimethicone (Shin-Etsu), silicones containing polyalkylene oxide groups, such as the commercially available emulsifier Silwet® 7001, manufactured by Momentive Performance Materials (Albany, NY), Dow Corning FG-10, Silwet® L-77 (polyalkylene oxide modified heptamethyl trisiloxane containing a methyl end group and 1 pendant group and having an average molecular weight of 645) and Silwet® L-7608 (polyalkylene oxide modified heptamethyl trisiloxane containing a hydrogen end group and one pendant group and having an average molecular weight of 630) available from Momentive Performance Materials; Lambent™ MFF-199-SW (containing a hydrogen end group and one pendant polyethylene oxide group and having an average molecular weight between 600 to 1000) available from Lambent Technologies Inc. (Gurnee, Illinois); silicone copolyol based carboxylate esters, such as SW-CP-K (containing a phthalate end group and one polyethylene oxide pendant group and having an average molecular weight between 800 and 1100) and Lube CPI (containing a phthalic acid end group and 3 to 5 pendant groups and having an average molecular weight between 2900 and 5300) available from Lambent Technologies Inc.; alkyl-dimethicone copolyol type surfactants, such as described in U.S. Pat. No. 7,083,800 (Terren et al, 2006), including such silicone emulsifiers commercially sold under the names “Abil® WE 09”, “Abil® WS 08” and “Abil® EM 90” (Evonik Industries AG, Essen, Germany) and cationic silicone emulsifiers, such as described in U.S. Pat. No. 5,124,466 (Azechi et al., 1992).

Exemplary cationic surfactants include but are not limited to homopolymers and copolymers derived from free radically polymerizable acrylic or methacrylic ester or amide monomers. The copolymers can contain one or more units derived from acrylamides, methacrylamides, diacetone acrylamides, acrylic or methacrylic acids or their esters, vinyl lactams such as vinyl pyrrolidone or vinyl caprolactam, and vinyl esters. Exemplary polymers include copolymers of acrylamide and dimethyl amino ethyl methacrylate quaternized with dimethyl sulfate or with an alkyl halide; copolymers of acrylamide and methacryloyl oxyethyl trimethyl ammonium chloride; the copolymer of acrylamide and methacryloyl oxyethyl trimethyl ammonium methosulfate; copolymers of vinyl pyrrolidone/dialkylaminoalkyl acrylate or methacrylate, optionally quaternized, such as the products sold under the name GAFQUAT™ by International Specialty Products; the dimethyl amino ethyl methacrylate/vinyl caprolactam/vinyl pyrrolidone terpolymers, such as the product sold under the name GAFFIX™ VC 713 by International Specialty Products; the vinyl pyrrolidone/methacrylamidopropyl dimethylamine copolymer, marketed under the name STYLEZE™ CC 10 by International Specialty Products; and the vinyl pyrrolidone and quaternized dimethyl amino propyl methacrylamide copolymers such as the product sold under the name GAFQUAT™ HS 100 by International Specialty Products; quaternary polymers of vinyl pyrrolidone and vinyl imidazole such as the products sold under the trade name Luviquat® (product designation FC 905, FC 550, and FC 370) by BASF; acetamidopropyl trimonium chloride, behenamidopropyl dimethylamine, behenamidopropyl ethyldimonium ethosulfate, behentrimonium chloride, cetethyl morpholinium ethosulfate, cetrimonium chloride, cocoamidopropyl ethyldimonium ethosulfate, dicetyldimonium chloride, dimethicone hydroxypropyl trimonium chloride, hydroxyethyl behenamidopropyl diammonium chloride, quaternium-26, quaternium-27, quaternium-53, quaternium-63, quaternium-70, quaternium-72, quaternium-76 hydrolyzed collagen, PPG-9 diethylammonium chloride, PPG-25 diethylammonium chloride, PPG-40 diethylmonium chloride, stearalkonium chloride, stearamidopropyl ethyl dimonium ethosulfate, steardimonium hydroxypropyl hydrolyzed wheat protein, steardimonium hydroxypropyl hydrolyzed collagen, wheat germamido-propalkonium chloride, wheat germamidopropyl ethyldimonium ethosulfate, polymers and copolymers of dimethyl diallyl ammonium chloride, such as Polyquaternium-4, Polyquaternium-6, Polyquaternium-7, Polyquaternium-10, Polyquaternium-11, Polyquarternium-16, Polyquaternium-22, Polyquaternium-24, Polyquaternium-28, Polyquaternium-29, Polyquaternium-32, Polyquaternium-33, Polyquaternium-35, Polyquaternium-37, Polyquaternium-39, Polyquaternium-44, Polyquaternium-46, Polyquaternium-47, Polyquaternium-52, Polyquaternium-53, Polyquarternium-55, Polyquaternium-59, Polyquaternium-61, Polyquaternium-64, Polyquaternium-65, Polyquaternium-67, Polyquaternium-69, Polyquaternium-70, Polyquaternium-71, Polyquaternium-72, Polyquaternium-73, Polyquaternium-74, Polyquaternium-76, Polyquaternium-77, Polyquaternium-78, Polyquaternium-79, Polyquaternium-80, Polyquaternium-81, Polyquaternium-82, Polyquaternium-84, Polyquaternium-85, Polyquaternium-87, PEG-2-cocomonium chloride, and mixtures thereof; polyalkyleneimines such as polyethyleneimines, polymers containing vinyl pyridine or vinyl pyridinium units, condensates of polyamines and epichlorhydrins; quaternary polyurethanes; salts of a primary, secondary, or tertiary fatty amine, optionally polyoxyalkylenated; a quaternary ammonium salt derivative of imidazoline, or an amine oxide; mono-, di-, or tri-alkyl quaternary ammonium compounds with a counterion such as a chloride, methosulfate, tosylate, including, but not limited to, cetrimonium chloride, dicetyidimonium chloride and behentrimonium methosulfate.

Exemplary anionic surfactants include, but are not limited to, one or more of a carboxylate such as, without limitation, alkylcarboxylates (e.g., carboxylic acid and/or its salts), polyalkoxycarboxylates (e.g., polycarboxylic acid and/or its salts), alcohol ethoxylate carboxylates, nonylphenol ethoxylate carboxylates, or combinations thereof; sulfonates such as, without limitation, alkylsulfonates, alkylbenzenesulfonates (e.g., dodecyl benzene sulfonic acid and/or its salts), alkylarylsulfonates, sulfonated fatty acid esters, or combinations thereof; sulfates such as, without limitation, sulfated alcohols, sulfated alcohol ethoxylates, sulfated alkylphenols, alkylsulfates, sulfosuccinates, alkylether sulfates, or combinations thereof; phosphate esters such as, without limitation, alkyl-phosphate esters; or combinations thereof. Exemplary anionic surfactants include sodium alkylarylsulfonate, alpha-olefinsulfonate, fatty alcohol sulfates and combinations thereof. Exemplary sulfosuccinates include alkyl sulfosuccinates and amido sulfosuccinates, such as disodium lauryl sulfosuccinate (CAS 26838-05-1), disodium laureth sulfosuccinate (CAS No. 39354-45-5), disodium oleamido MIPA sulfosuccinate (CAS No. 67815-88-7), and combinations thereof.

Exemplary amphoteric surfactants (or zwitterionic surfactants) include, but are not limited to, imidazoline derivatives, betaines, imidazolines, sultaines, propionates, amine oxides or combinations thereof, including imidazolinium betaine, dimethylalkyl lauryl betaine, alkylglycine, and alkyldi(aminoethyl)glycine. The betaines can be an alkyl betaine, an alkylamido betaine, or a mixture thereof, such as any one of cetyl betaine (CAS No. 693-33-4), lauryl betaine (CAS No. 683-10-3), cocamidopropyl betaine (CAS No. 61789-40-0), lauramidopropyl betaine (CAS No. 4292-10-8), or a combination thereof.

The surfactant can be a linear alcohol ethoxylate (e.g., Tomadol® 25-7, available from Evonik Industries AG, Essen, Germany), or a C11 alcohol ethoxylate 5 E.O. (e.g., Tomadol® 1-5, Evonik), or a C11 alcohol ethoxylate 7 E.O. (e.g., Tomadol® 1-7, Evonik), or a C11 alcohol ethoxylate 9 E.O. (e.g., Tomadol® 1-9, Evonik), or sodium lauryl sulfate, or a sodium dodecyl benzene sulfonate (e.g., available from Stepan Company, Northfield, IL), or a C9-C11 alcohol ethoxylate (e.g., Tomadol® 91-6, Evonik), or a C12-C18 alcohol ethoxylate (e.g., available from Croda Inc., Mill Hall, PA), or PEG 7 glyceryl cocoate, or a sodium lauroyl sarcosinate (e.g., Perlastan® L30 available from Schill and Seilacher GmbH, Boblingen, Germany), or any combination thereof. The surfactant can include 2-butenedioic acid-1-dodecyl ester (CAS No. 2424-61-5), sodium 2-sulfo-butanedioic acid (CAS No. 13419-59-5), sodium lauroyl sarcosinate (CAS No. 137-16-6), or a combination thereof.

Biosurfactants also can be used. The biosurfactant can be a polymeric biosurfactant, a glycolipid, a lipopeptide, a lipoprotein, a phospholipid, a flavolipid, or a combination thereof. The biosurfactant can be a lecithin, a saponin, a rhamnolipid, a sophorolipid, a mannosylerythritol lipid, a marine alga glycolipid, a glucose lipid, a cellulose lipid, a trehalose lipid, an alkyl glucoside, a cellobiose lipid, a polyol lipid, a protein polyamine, a lipopolysaccharide, fengycin, iturin, lichenysin, surfactin, or a combination thereof.

The effervescent solid dosage forms provided herein can include a fragrance, alone or in combination with an additional component, such as a surfactant, carbonate, bicarbonate, or combinations thereof. The effervescent solid dosage forms provided herein can include a carbonate or bicarbonate or a combination thereof, alone or in combination with an additional component, such as a surfactant, fragrance, or a combination thereof. The effervescent solid dosage forms provided herein can include a chelant, alone or in combination with an additional component. The effervescent solid dosage forms provided herein can include a buffer, alone or in combination with an additional component. The effervescent solid dosage forms provided herein can include a phosphate, alone or in combination with an additional component. The effervescent solid dosage forms provided herein can include a wetting agent or a dispersing agent or both, alone or in combination with an additional component. The effervescent solid dosage forms provided herein can include an acetate, alone or in combination with an additional component. The acetate can be a sodium acetate, potassium acetate, magnesium acetate, calcium acetate, or any combination thereof. The effervescent solid dosage forms provided herein can include an enzyme, alone or in combination with an additional component. The enzyme can be a lipase, a protease, a peroxidase, an oxidase, an amylolytic enzyme, a cellulase, a polyesterase, a glucanase, an amylase, a glucoamylase, a glycosidase, a hemicellulase, a mannanase, a xylanase, a xyloglucanase, a pectinase, a β-glucosidase, or any combination thereof.

The effervescent solid dosage forms provided herein can include a humectant, alone or in combination with an additional component. Any humectant known in the art can be included in the effervescent solid dosage forms. The humectant can be selected from the group consisting of glycerin, diglycerin, triacetin (glycerol triacetate), 1,3-butylene glycol, pentylene glycol, polyethylene glycol having a weight-average molecular weight of 800 or less, dipropylene glycol, panthenol, hyaluronic acid, sodium hyaluronate, and mixtures thereof. The humectant can be present in the effervescent solid dosage forms provided herein in the range of about 0.1 wt % to 10 wt %, or in the range of about 0.25 wt % to 7.5 wt %, or in the range of about 0.5 wt % to 5 wt %, based on the weight of the solid dosage form.

The effervescent solid dosage forms provided herein can include an emollient, alone or in combination with a humectant and/or an additional component. Any emollient known in the art can be included in the effervescent solid dosage forms. The emollient can be selected from the group consisting of lanolin oil, cetyl alcohol, cetearyl alcohol, lauryl alcohol, oleyl alcohol, stearyl alcohol, PPG-15 stearyl ether, octyldodecanol, diisopropyl adipate, cetearyl octanoate. glycerin mono-, di-, and tri-esters, cholesterol, cholesterol esters, a silicone fluid such as a linear silicone having a viscosity of from about 10 cP to about 100,000 cP at 25° C. or a cyclomethicone, a light mineral oil, a petrolatum, a coconut oil, isopropyl myristate, soybean oil, stearic acid, caprylic acid, oleic acid, palmitic acid, lauric acid, octylpalmitate, octyl stearate, glyceryl stearate, and combinations thereof. The emollient can be present in the effervescent solid dosage forms provided herein in the range of about 0.1 wt % to 10 wt %, or in the range of about 0.25 wt % to 7.5 wt %, or in the range of about 0.5 wt % to 5 wt %, based on the weight of the solid dosage form.

The effervescent solid dosage forms provided herein can include a foam booster, alone or in combination with an additional component. Any foam booster known in the art can be included in the effervescent solid dosage forms. The foam booster can be selected from among a polyethylene glycol such as for example PEG-75 or PEG-100, aliphatic alcohols, alkanolamides, and amine oxides. Examples of the aliphatic alcohol include aliphatic alcohols having 8 to 18 carbon atoms, preferably 10 to 16 carbon atoms, and more preferably 12 to 14 carbon atoms. Examples of the alkanolamides include monoethanolamide, diethanolamide, and N-methylethanolamide. Examples of the amine oxide include mono long chain alkyl amine oxides having a long chain length alkyl group having 8 to 18 carbon atoms, preferably 12 to 14 carbon atoms, and an alkyl group having 1 to 3 carbon atoms. Exemplary foam boosters include glycerylmonocaprylate, cocamidopropyl hydroxysultaine, sorbitan sesquicaprylate, dodecyl alcohol, alkanolamide, monoethanolamide, diethanolamide, N-methyl ethanol amide, lauramidopropyl betaine cocoamidopropyl betaine. The foam booster can be present in the effervescent solid dosage forms provided herein in the range of about 0.01 wt % to 15 wt %, or in the range of about 0.05 wt % to 10 wt %, or in the range of about 0.1 wt % to 7.5 wt %, or in the range of about 0.25 wt % to 5 wt %, based on the weight of the solid dosage form.

The additional component can be present in the effervescent solid dosage forms provided herein in the range of about 0.05 wt % to 50 wt %, or in the range of about 0.1 wt % to 45 wt %, or in the range of about 0.25 wt % to 40 wt %, or in the range of about 0.5 wt % to 35 wt %, based on the weight of the solid dosage form.

In some embodiments, the effervescent solid dosage forms provided herein are free of EDTA. In some embodiments, there is no calcium salt of EDTA or magnesium salt of EDTA in the effervescent solid dosage forms. In some applications, the effervescent solid dosage forms provided herein are free of borates, boric acid or perborates.

5. Forms

The effervescent solid dosage forms provided herein are provided in a dry form. The dry form is shelf stable for extended periods of time, and readily can be dissolved in a solvent, such as water, to produce a clean and/or disinfecting solution. The effervescent solid dosage form compositions provided herein can be provided in any dry form known in the art, e.g., as an agglomerate, granule, tablet, capsule, pellet, puck, brick, briquette, block, layered compression, or composite. Any one of the agglomerate, granule, tablet, capsule, pellet, puck, brick, briquette, block, layered compression, or composite can be dissolved in a solvent to provide the composition in the form of a liquid.

Any appropriate solvent can be used to dissolve the effervescent solid dosage form composition to provide it in liquid form. In some embodiments, the solvent is predominately water. The water can be deionized water, distilled water, hard water, city water, well water, water supplied by a municipal water system, water supplied by a private water system, or treated water. In general, hard water refers to water having a level of calcium and/or magnesium ions in excess of about 100 ppm. For example, hard water containing 400 ppm calcium carbonate can be used to dissolve the solid forms of the compositions provided herein.

In some applications, the solution is substantially aqueous, meaning that while the majority of the solvent in the liquid form is water, non-water solvents can be present. In some applications, the solution produced by dissolution of the effervescent solid dosage form compositions provided herein contains a non-water solvent in an amount that its less than about 25 wt % of the resulting solution, but more than 0.01 wt % of the resulting solution. In some applications, the solvent used to dissolve the effervescent solid dosage form compositions provided herein contains a non-water solvent in an amount from about 0.5 wt % to about 10 wt % based on the weight of the solution. In some applications, the solvent used to dissolve the effervescent solid dosage form compositions provided herein contains a non-water solvent in an amount from about 1 wt % to about 25 wt %, or from about 0.5 wt % to about 10 wt %, or from about 0.05 wt % to about 5 wt %, based on the weight of the solution. It is to be understood that all values and ranges between these values and ranges are encompassed by the compositions and methods provided herein.

The effervescent solid dosage form compositions provided herein can dissolve in a solvent to form a clear solution.

6. Protective Layer

The effervescent solid dosage form compositions provided herein can include a protective layer. The protective layer protects the composition from the influence of physical or chemical action applied from the surroundings. The protective layer can be on or around or encapsulating the effervescent solid dosage form compositions provided herein. In some embodiments, the protective layer can be in the form of a film, packet, pouch, sheath or envelope that surrounds the effervescent solid dosage form composition. The protective layer can contain or be made of a material that dissolves or disperses rapidly when exposed to water, thereby releasing the contained effervescent solid dosage form composition to the water.

For example, the protective layer can be made of or contain any film forming water soluble polymer, such as water soluble polyvinyl alcohol, polyvinylpyrrolidone, cellulose derivatives such as sodium cellulose acetate propionate sulfate and hydroxypropyl methyl cellulose (HPMC), xanthan gum, alginate, gellan gum, gelatin, modified starch or any combination thereof. Methods of preparing water soluble or dispersible pouches are described, e.g., in U.S. Pat. No. 2,760,942 (Oakley, 1956); U.S. Pat. No. 3,086,007 (Touey et al., 1963); U.S. Pat. No. 3,198,740 (Dunlop, Jr. et al., 1965); U.S. Pat. No. 3,413,229 (Bianco et al., 1968); U.S. Pat. No. 3,892,905 (Albert, 1975); U.S. Pat. No. 4,155,971 (Wysong, 1979); U.S. Pat. No. 4,340,491 (lee, 1982); U.S. Pat. No. 4,416,791 (Haq, 1983); and U.S. Pat. No. 4,626,372 (Kaufmann et al., 1986); and in WO2008/087424. The thickness of the protective layer can be any appropriate thickness. For example the protective layer can have a thickness of from about 10 μm to about 2500 μm thick. The protective layer can be prepared so that when applied to the effervescent solid dosage form compositions provided herein, the final dried protective layer adds from at or about 0.01 wt % to at or about 10 wt % based on the weight of the composition. The protective layer can be in the form of a water soluble or dispersible protective pouch. The thickness of the film used to produce the pouch can be up to 5 mm, but can be 2 mm or less, or 1 mm or less, and can be 25 μm to 250 μm thick.

The protective pouch can be used to provide unit dosages of the effervescent solid dosage form compositions, pre-weighed for addition to a predetermined amount of solvent, such as water. For example, the protective pouch can contain an amount of effervescent solid dosage form composition provided herein to make a spray bottle solution of a hard surface sanitizing or disinfectant spray when dissolved in water, or an amount that makes a sanitizer or disinfectant for mop buckets when added to a standard industrial bucket, or a laundry sanitizer or disinfectant when added to the water in a washing machine, or surgical instrument sanitizer when added to water in an instrument sterilizing tray, or a dishwasher sanitizer or cleaner when included in a wash cycle of a dishwashing machine.

A polymer coating also can be applied to the surface of the effervescent solid dosage form composition as a protective layer. Any polymer coating known in the art can be used. Suitable coating materials can include adipic acid, azelaic acid, glutaric acid, malonic acid, oxalic acid, pimelic acid, sebacic acid, suberic acid, succinic acid, undecanedioic acid, dodecanedioic acid, tridecanedioic acid, hydroxypropyl cellulose, hydroxypropyl methylcellulose (e.g., Opadry ° coating), polyvinylacetate, hydroxyethyl cellulose, methylhydroxyethyl cellulose, methyl cellulose, ethyl cellulose (e.g., Surelease ° coating), cellulose acetate, sodium carboxymethyl cellulose, polymers and copolymers of acrylic acid and methacrylic acid and esters thereof (e.g., Eudragit® RL, Eudragit® RS, Eudragit® L100, Eudragit® S100, Eudragit® NE), starch, modified starch, maltodextrin, a wax, gum arabic, shellac, water soluble polyvinyl alcohol, polyalkylene glycols, acrylic polymer, such as sodium polyacrylate, or polyvinylpyrrolidone, or combinations thereof. In some embodiments, the polymer coating is or contains a water soluble polyvinyl alcohol or a polyalkylene glycol. Exemplary polyalkylene glycols include polyethylene glycol and polypropylene glycol. When used, the molecular weight of the polyalkylene glycol is selected to be in the range of about 400 to about 8000.

When present, the polymer coating can be applied to any desired thickness or weight gain. In some embodiments, the weight gain due to the application of a protective polymer is from about 0.1 wt % to about 10 wt %, or from about 0.5 wt % to about 5 wt %, or from 1 wt % to about 8 wt %. It is to be understood that all values and ranges between these values and ranges are encompassed by the compositions and methods provided herein.

The polymer coating as a protective layer can be applied to the effervescent solid dosage form composition provided herein using any appropriate method known in the art. For example, a polymer coating can be applied by conventional coating techniques such as, e.g., pan coating, fluidized bed coating, fluidized bed bottom sprayed coating, air suspension coating, compression coating, spray drying, spray congealing, solvent evaporation, melting, coacervation, or interfacial complexation or any combination thereof. The polymer solution or suspension used to form the polymer coating can be in a conventional coating pan, or, alternatively, using an automated system such as a Fluidized Bed Processor (e.g., those available from Glatt Air Techniques Inc., Ramsey, NJ) or a top spray or bottom spray fluid bed coaters (e.g., Precision Coater™, available from Niro Inc., Columbia, MD).

The protective layer of the effervescent solid dosage form compositions provided herein, whether in the form of a water soluble or dispersible pouch or as a polymer coating on the surface of the effervescent solid dosage form composition, provides a means for minimizing human contact with the components of the effervescent solid dosage form composition. This makes the effervescent solid dosage form compositions provided herein safer to handle by consumers, keeping the consumer from directly contacting the components of the composition, reducing possible interaction with exposed skin and eyes.

### D. Methods of Preparation

1. Preparation of the Effervescent Solid Dosage Form Compositions

The effervescent solid dosage form compositions provided herein can be prepared by blending together the preservative, the sugar acid lactone, the effervescence generator, and any additional components, to form a mixture in which the powders are evenly distributed and homogeneous. Any powder blending technique that results in a uniform final product can be used. Known devices, such as a Hobart® planetary mixer, a vee-blender, a vee-cone blender, a rotary batch mixer, a fluidized bed mixer, a ribbon blender, a paddle blender and a plow blender or combinations thereof, can be used to mix the components. The mixing can be carried out at room temperature under atmospheric pressure, and is not adversely affected by temperature or pressure conditions. It is possible that high humidity can a negative impact on the blending. A dehumidification system can be used in the blending area to maintain a relative humidity of about 25% or less, or 15% or less, if desired. Any dehumidification system known in the art can be used to control humidity (e.g., any of the dehumidification systems available from Munters AB, Kista, Sweden). The amount of time required to form a uniform blend can depend on the amount of material to be blended and the size and type of mixing equipment selected. The components of the effervescent solid dosage form compositions provided herein are not adversely affected by the time of mixing. In some embodiments, a vee-cone blender large enough so that no more than 50% of its capacity is used to contain the components is used to mix the components for 1 hour to obtain a uniform mixture.

2. Preparation of Agglomerates or Granules

In some embodiments, the uniform blend of the components of the effervescent solid dosage form composition can be agglomerated to form larger-sized agglomerates or granules. In some embodiments, the agglomerates or granules are free-flowing. The uniform blend can be agglomerated using any granulation process, such as wet granulation, dry granulation, fluid bed granulation, or a combination thereof, either alone or in the presence of an excipient such as a binder. Any binder that is effective in forming the agglomerate and creating a stable agglomerated structure can be selected. Exemplary binders include polyvinyl alcohol, polyethylene glycol, an alcohol, anionic and nonionic surfactants, film forming polymers, fatty acids, fatty acid polyol esters, polyglycols, hexitols, and fatty alcohol oxyalkylates and combinations thereof.

Agglomerates can be prepared using any conventional agglomeration equipment that facilitates mixing and intimate contacting of a liquid binder with the components of the antimicrobial composition such that it results in agglomerated particles. The agglomerated particles can take the form of flakes, prills, noodles, ribbons, agglomerates or granules. In some embodiments, a preferred form is a granule. Suitable agglomerators for use in the production of agglomerates include vertical agglomerators (e.g. Schugi Flexomix or Bepex Tirboflex), rotating drums, and pan agglomerators.

In some applications, the effervescent solid dosage form compositions provided herein, can be formed into a tablet, or filled into a capsule, or be provided in a dissolvable pouch, in order to provide a unit dosage form of the effervescent solid dosage form compositions provided here.

3. Tablet Preparation

In some applications, the effervescent solid dosage form compositions provided herein can be formed into a tablet. Tablets have several advantages over powdered products: they do not require measuring and are thus easier to handle and dispense for preparation of a disinfectant or cleaning solution, and they are more compact, facilitating more economical storage and reducing shipping costs.

A tablet form of the effervescent solid dosage form compositions provided herein can be of any geometric shape. Exemplary shapes include spherical, cube, disk, rod, triangular, square, rectangular, pentagonal, hexagonal, lozenge, modified ball, core rod type (with hole in center), capsule, oval, bullet, arrowhead, compound cup, arc triangle, arc square (pillow), diamond, half-moon and almond. The tablets can be convex or concave. The tablets can be flat-faced plain, flat-faced bevel-edged, flat-faced radius edged, concave bevel-edged or any combination thereof. In some embodiments, the tablet can have a generally axially-symmetric form and can have a round, square or rectangular cross-section. The tablet can be of uniform composition, or can contain two or more distinct regions having differing compositions

Tablets containing the effervescent solid dosage form composition provided herein can be prepared using any method known in the art, including compression, casting, briquetting, injection molding and extrusion. In some embodiments, the tablet preferably is produced by compression, for example in a tablet press. Direct compression often is considered to be the simplest and the most economical process for producing tablets. Direct compression requires only two principal steps: the mixing of all the ingredients and compressing this mixture into a tablet. Any method known in the art for formation of a tablet can be used to prepare the effervescent solid dosage form composition into a tablet. For example, the components of the effervescent solid dosage form composition can be prepared by admixing the components to achieve a uniform mix. Any powder blending, mixing or shaking technique that results in a uniform final product can be used. Known devices, such as a Hobart® planetary mixer, a vee-blender, a vee-cone blender, a rotary batch mixer, a fluidized bed mixer, a ribbon blender, a paddle blender and a plow blender or combinations thereof, can be used to mix the components. The uniform blend can be blended with lubricants or other excipients prior to tableting.

The resulting uniform mix then can be placed into a die of the desired geometry in a conventional tablet press, such as a single stroke or rotary press. The press includes a punch suitably shaped for forming the tablet. The uniform mix is then subjected to a compression force sufficient to produce a tablet, and a tablet containing the antimicrobial composition provided herein is ejected from the tablet press. Agglomerates and granules also can be used to form tablets. The agglomerates or granules can be blended with lubricants or other excipients prior to tableting.

Any tableting equipment known in the art can be used for tablet formation. Suitable equipment includes a standard single stroke or a rotary press. Such presses are commercially available, and are available from, e.g., Carver, Inc. (Wabash, IN), Compression Components & Service, LLC (Warrington, PA), Specialty Measurements Inc. (Lebanon, NJ), GEA Pharma Systems (Wommelgem, Belgium), Korsch America Inc. (South Easton, MA) or Bosch Packaging Technology (Minneapolis, MN).

The tablets form of the effervescent solid dosage form compositions provided herein can have any desired diameter, such as a diameter of between about 5 mm and about 75 mm. In some embodiments, the tablets have a diameter of at least 6 mm, at least 7 mm, at least 8 mm, at least 9 mm, at least 10 mm, at least 15 mm, at least 20 mm, at least 25 mm, at least 30 mm, at least 35 mm, at least 40 mm, at least 45 mm, at least 50 mm, at least 55 mm, at least 60 mm or at least 70 mm. The tablets can be of any weight, such as a weight between 100 mg and 100 g. In some embodiments, the tablet can have a total weight of about 1 g to about 30 g, or from about 5 g to about 25 g, or from about 10 g to about 30 g. It is to be understood that all values and ranges between these values and ranges are encompassed by the compositions and methods provided herein.

The tableting can be carried out at room temperature under atmospheric pressure, and is not adversely affected by temperature or pressure conditions. High humidity can have a negative impact on tableting. A dehumidification system can be used in the tableting area to maintain a relative humidity of about 25% or less, or 15% or less. Any dehumidification system known in the art can be used to control humidity (e.g., any of the dehumidification systems available from Munters AB, Kista, Sweden).

The compression force can be selected to most economically provide optimum tablet integrity and strength (measured, e.g., by tablet hardness), and having the desired product aesthetics and dissolution characteristics. For example, the tablet can be compressed by applying a compression force of at least about 1 tf (ton-force (metric)). The tablet can be compressed by applying a compression force in a range of at least about 1 tf (ton-force (metric)) to about 20 tf (ton-force (metric)). The compression force can be 1 metric ton to 15 metric tons (1 tf to 15 tf), or 5 metric tons to 15 metric tons, or 5 metric tons to 20 metric tons, or 10 metric tons to 20 metric tons. It is to be understood that all values and ranges between these force ranges are encompassed by the compositions and methods provided herein.

### E. Packaged Systems

The effervescent solid dosage form composition can be provided in a packaging material to form a packaged system. The packaging material can be rigid or flexible, and can be composed of any material suitable for containing the effervescent solid dosage form compositions provided herein. Examples of suitable packaging materials include glass, metal foil, treated metal foil, metal foil pouches, plastic, plastic film, plastic sheets, blister packs, cardboard, cardboard composites, paper and treated paper, and any combination thereof.

The packaged system can include a container for dissolving the effervescent solid dosage form in a solvent to form an antimicrobial composition solution, or a receptacle for containing and/or dispensing the formulation and solvent, or both a container and a receptacle. In some embodiments, the receptacle can be used for preparing, dispensing and storing the solution of the dissolved effervescent solid dosage form. Any receptacle capable of containing the solution of the dissolved effervescent solid dosage form can be included in the packaged system. In particular, the receptacle can include a spray bottle, a sponge, a conventional hand sprayer container, an electric spray dispenser container (see U.S. Pat. No. 5,716,007 (Nottingham et al, 1998) and U.S. Pat. No. 5,716,008 Nottingham et al, 1998), a bucket, a can, a drum, a towelette, a wipe, or a pad or any combination thereof.

### F. Articles of Manufacture

The effervescent solid dosage form compositions provided herein can be part of an article of manufacture, which can include a container the effervescent solid dosage form, such as for shipping and/or storage. The effervescent solid dosage form compositions provided herein can be stored or shipped in a variety of containers, and the containers can be made of or contain any of a variety of container materials, such as glass, acrylonitrile butadiene styrene (ABS), high impact polystyrene, polycarbonate, high density polyethylene, low density polyethylene, high density polypropylene, low density polypropylene, polyethylene terephthalate, polyethylene terephthalate glycol and polyvinylchloride and combinations thereof. The containers can include barrier films to increase storage stability. Suitable barrier films can include nylons, polyethylene terephthalate, fluorinated polyethylenes, and copolymers of acrylonitrile and methylmethacrylate.

An article of manufacture can include an effervescent solid dosage form composition provided herein and a set of instructions, such as for the use of the effervescent solid dosage form compositions. In some applications, the article of manufacture includes instructions for preparing a cleaning/disinfectant solution by dissolving one or more of the effervescent solid dosage form compositions provided herein in an appropriate solvent. The article of manufacture can include one of the effervescent solid dosage form compositions provided herein and storage instructions, or a material safety data sheet or a combination thereof. The article of manufacture can include one of the effervescent solid dosage form compositions provided herein and a dispenser or applicator for preparing or for use with the cleaning or disinfectant solution prepared by dissolution of the effervescent solid dosage form composition, alone or in combination of any of storage instructions, preparation instructions or a material safety data sheet.

### G. Applications

One or more of the effervescent solid dosage form compositions provided herein can be added to a predetermined amount of solvent to prepare a disinfecting, cleaning, or sanitizing solution. For example, an effervescent solid dosage form provided as a compressed tablet can be used to make a solution when dissolved in a solvent of a hard surface cleaning, sanitizing or disinfectant spray, or a cleaner, sanitizer or disinfectant for mop buckets, or a laundry sanitizer or disinfectant, or a laundry machine cleaner, sanitizer or disinfectant, or a solution that cleans, sanitizes or disinfects industrial or household appliances, or a solution that cleans or disinfects surgical instruments, or a solution that cleans or disinfects dental instruments, or a solution that cleans or disinfects cosmetological instruments or equipment, or a cleaning or sanitizing liquid hand soap formulation, or a foaming hand soap formulation for cleaning or sanitizing a skin surface of the hands, or a solution for cleaning or sanitizing a skin surface, or a solution for cleaning or sanitizing an article of clothing, or a multi-surface cleaning, disinfecting, or sanitizing solution, or a degreaser spray, or a personal care formulation.

The amount of preservative included in the effervescent solid dosage form compositions can be calculated to yield a solution when dissolved in a solvent containing 50 to 50,000 mg/L. In some formulations, the amount of sodium benzoate or potassium sorbate included in the effervescent solid dosage form compositions result in solutions, when the effervescent solid dosage form compositions is dissolved in a solvent, having concentrations of sodium benzoate and/or potassium sorbate of about 0.025 wt % to 1 wt % based on the total weight of the solution, as these concentrations have been found to be generally sufficient to preserve a liquid product, depending on the formulation. In some formulations, the amount of sodium benzoate and/or potassium sorbate included in the effervescent solid dosage form compositions result in solutions, when dissolved in a solvent, having concentrations of sodium benzoate and/or potassium sorbate of about 0.02 wt % to 1.0 wt %, or about 0.025 wt % to 0.9 wt % based on the total weight of the solution. In some formulations, the amount of sodium benzoate and/or potassium sorbate included in the effervescent solid dosage form compositions result in solutions, when dissolved in a solvent, having concentrations of sodium benzoate and/or potassium sorbate of about 0.02 wt % to 1.0 wt %, or about 0.05 wt % to about 0.8 wt % based on the total weight of the solution, or about 0.075 wt % to about 0.75 wt % based on the total weight of the solution, or about 0.1 wt % to about 0.7 wt % based on the total weight of the solution. It is to be understood that all values and ranges between these values and ranges are encompassed by the compositions and methods provided herein.

When the effervescent solid dosage form compositions are dissolved in a solvent to form a solution, the pH of the resulting solution can be in the range of 3.0 to 6.0, or in the range of 3.5 to 6.0, or in the range of 4.0 to 6.0, or in the range of 4.5 to 6.0, or in the range of 5.0 to 6.0, or in the range of 4.0 to 5.7, or in the range of 4.1 to 5.6, or in the range of 4.3 to 5.5, or in the range of 4.5 to 5.4, or in the range of 4.7 to 5.3.

Dissolution of the effervescent solid dosage form compositions provided herein in a solvent can produce cleaning, sanitizing or disinfectant solutions. The cleaning, sanitizing or disinfectant solutions can be applied in any situation where it is desired to clean, sanitize or disinfect a surface, such as a skin surface, the surface of cloth or clothing, or a hard surface. The cleaning, sanitizing or disinfectant solutions are particularly well suited for treating hard surfaces. Such hard surfaces can be found in private households as well as in commercial, medical, institutional and industrial environments. The hard surfaces can be made of or contain any number of different materials, e.g., enamel, ceramic, glass, stainless steel, chrome, vinyl, linoleum, melamine, glass, fiberglass, Formica®, granite, marble, hardwood, grout, porcelain, concrete, plastic, plastified wood, metal or any painted or varnished or sealed surface. The sanitizing or disinfectant solutions can contain a combination of components that can clean, sanitize or disinfect the hard surface within 1 to 30 minutes of application, or within 5 to 30 minutes of application.

Exemplary hard surfaces include, but are not limited to, bathroom surfaces (e.g., floor, drains, tub, shower, mirrors, sinks, toilet, toilet seat, urinal, bidet, lavatory pans, countertops, shower doors or curtains, shower stalls, wash basins, bathroom fixtures, windows, fans, walls, light fixtures and tiles); appliance surfaces (e.g., coffee maker, stove, oven, range, sink, garbage disposal, dishwashers, refrigerator, freezer, microwave, toaster, mixers, washing machine, dryer, barbeque); kitchen surfaces (e.g., appliances, floor, fixtures, light fixtures, fans, countertops, crockery, cupboards, cutlery, doors, door handles, walls, tables, chairs, cabinets, drawers, food processing equipment, flatware, utensils, floors, glassware, phones, clocks, plate ware, shelves, pantry, sinks, dishwashers, windows, and work surfaces); transportation devices (e.g., cars, bicycles, snowmobiles, motorcycles, off-road-vehicles, tractors, recreation vehicles, boats, and planes); yard equipment; farm equipment; laboratory surfaces (e.g., autoclaves, work surfaces, hoods, clean rooms, storage rooms, cold rooms, countertops, centrifuges, and floors); computer surfaces (keyboards, monitors, housing, towers, laptops, and cables); hand rails; banisters; dental equipment or devices; medical devices or equipment; patient care equipment; patient monitoring equipment; surgical devices or equipment or instruments; veterinarian equipment; tools; and utility devices (e.g., telephones, radios, televisions, entertainment centers, stereo equipment, CD and DVD players, play stations, and analog and digital sound devices). Countertops can include tile surfaces, granite, marble or other stone surfaces, Corian® or other manmade hard surfaces, engineered quartz such as Viatera® quartz surfaces (LG Hausys), wood surfaces, glass surfaces, acrylic or polyester resin surfaces, concrete surfaces and stainless steel surfaces.

In some applications, the effervescent solid dosage form compositions provided herein can include a metal protectant. In some embodiments, the metal protectant is a corrosion inhibitor. Exemplary corrosion inhibitors include C4-C16 alkyl pyrrolidones, C1-C18 alkylamines, benzoates, azoles, imidazoles, diazoles, triazoles, aromatic triazoles and their salts, such as benzotriazole, tolyltriazole, sodium tolyltriazole, monosodium phosphate, disodium phosphate, sodium hexametaphosphate, and potassium equivalents thereof, hydroxyethylidine di-phosphonic acid, 8-hydroxy-quinoline, orthophenylphenol, sarcosine, and sarcosinate corrosion inhibitors.

Preferred metal protectants include sarcosine and sarcosinate corrosion inhibitors. Sarcosinate corrosion inhibitors include sarcosine and salts thereof, and sarcosinate surfactants, and any combination thereof. Exemplary sarcosinate corrosion inhibitors include cocoyl sarcosine, lauroyl sarcosine, myristoyl sarcosine, N-acyl sarcosine, oleoyl sarcosine, and stearoyl sarcosine and the sodium salts, potassium salts or amine salts thereof, and sarcosinate surfactants or any combination thereof. In some embodiments, the corrosion inhibitor can include sodium cocoyl sarcosinate, sodium lauroyl sarcosinate, sodium myristoyl sarcosinate, sodium N-acyl sarcosinate, sodium oleoyl sarcosine, or sodium stearoyl sarcosine or any combination thereof.

The compressed tablet of the effervescent solid dosage form compositions provided herein can be of any size, such as from about 1 gram to about 25 grams, and one or more than one tablet can be used to make a solution when dissolved in a solvent. The solvent can include water, an alcohol, an aldehyde, a ketone, or a combination thereof. In some applications, the solvent is or contains water. A sufficient number of tablets can be added to a quantity of solvent to yield a solution having the desired concentration. The resulting solution can include from about 50 wt % to about 99.95 wt % of a solvent, and from about 0.05 wt % to about 50 wt % of the effervescent solid dosage form compositions provided herein dissolved in the solvent. The final concentration of the effervescent solid dosage form composition with respect to the total weight of the resulting solution can from about 0.05 wt % to about 50 wt % based on the weight of the resulting solution, or from about 0.1 wt % to about 20 wt % based on the weight of the resulting solution, or from about 0.5 wt % to about 10 wt % based on the weight of the resulting solution. It is to be understood that all values and ranges between these values and ranges are encompassed by the compositions and methods provided herein. For example, the amount effervescent solid dosage form composition with respect to the total weight of the resulting solution can be 0.05 wt %, 0.1 wt %, 0.5 wt %, 0.75 wt %, 1 wt %, 1.25 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt % 11 wt %, 11.5 wt %, 12 wt %, 12.5 wt %, 13 wt %, 13.5 wt %, 14 wt %, 14.5 wt %, 15 wt %, 20 wt %, 25 wt %, 30 wt %, 35 wt %, 40 wt %, 45 wt %, or 50 wt %.

For example, a 20 g tablet of an effervescent solid dosage form hand soap composition can be dissolved in 250 mL of water to yield a total concentration of about 7.4 wt % based on the total weight of the solution. A 25 g tablet of an effervescent solid dosage form hand soap composition can be dissolved in 225 mL of water to yield a total concentration of about 10 wt % based on the total weight of the solution.

Application Methods

The solutions prepared by dissolving the effervescent solid dosage form compositions provided herein in a solvent can be applied to surfaces by any technique or method known in the art. Exemplary application methods include spraying, wiping, direct application, immersion, or as part of a normal cleaning process, such as part of a laundry washing or dishwashing process. The solution can be applied directly to a surface as a spray or fine mist, via a woven or nonwoven substrate, brush, sponge, wipe or cleaning pad, or any combination thereof.

Also provided are methods of cleaning, disinfecting, or sanitizing a surface. The methods include contacting the surface with a liquid containing a dissolved effervescent solid dosage form composition provided herein. The method also can include dissolving the effervescent solid dosage form composition in a solvent. The solvent can include water, an alcohol, an aldehyde, a ketone, or a combination thereof. In some embodiments, the solvent is or contains water.

The methods provided herein can result in the destruction of, or prevention of the growth of, a microbe on a surface. The surface can be a skin surface or a hard surface. The microbe can be any one or a combination of a bacteria, archaea, unicellular and filamentous algae, unicellular and filamentous fungi (such as yeasts and molds), unicellular and multicellular parasites, and viruses.

The methods provided herein can result in killing bacteria on the treated surface, for example to prevent the spread of the bacteria. The methods also can inhibit the growth of bacteria on a treated surface. Exemplary bacteria that could be treated with a cleaning or disinfecting solution containing an antimicrobial composition provided herein include Acinetobacter, Burkholderia, Campylobacter, Clostridium, Enterococcus, Escherichia, Helicobacter, Klebsiella, Legionella, Listeria, Meningococcus, Mycobacterium, Pseudomonas, Salmonella, Shigella, Staphylococcus and Streptococcus.

### H. Examples

The following examples are included for illustrative purposes only and are not intended to limit the scope of the embodiments provided herein.

### Example 1—Multi-Surface Cleaner

| Component | Component (wt %) |
|---|---|
| Sodium carbonate¹ | 26 |
| Sodium percarbonate² | 5 |
| C11 alcohol ethoxylate 7 E.O.³ | 3 |
| GDL⁴ | 7 |
| Citric acid⁵ | 50 |
| Sodium benzoate⁶ | 7 |
| Fragrance | 2 |

¹= available from FMC Corporation, Philadelphia, PA, USA
²= available from Solvay Chemicals, Galveston, TX, USA
³= available from Evonik Industries AG, Essen, Germany
⁴= available from Jungbunzlauer Suisse AG, Basel, Switzerland
⁵= available from S.A. Citrique Belge N.V., Tienen, Belgium
⁶= available from Emerald Kalama Chemical, Kalama, WA. USA

To prepare the effervescent solid dosage form composition, each of the indicated amounts of the components was placed in a laboratory scale vee-cone blender (Munson Machinery Company, Inc., Utica, NY). The components were blended for 5 minutes to achieve a homogeneous blend.

The homogeneous blend was used to prepare compressed tablets. A 7 gram aliquot of the homogeneous blend was weighed to be made into a compressed tablet. The 7 gram blend was compressed into a tablet using a 15/16″ round tool and die. Tablet compression was performed using a CARVER Press (Carver, Inc. (Wabash, IN)) and pressed at a gauge reading of 7 metric tons (7 tf (ton force-metric)). All of the tablets exhibited smooth face surfaces, had good edges and good sidewalls with few defects.

Aqueous solutions of the compositions were prepared as follows. The compressed tablet was dissolved in 500 mL of tap water (without stirring) at 22° C. The dissolution time for the tablet to completely dissolve was 15 minutes. The resulting solution had a pH of 5.3 and the solution was clear.

### Example 2—Degreaser Spray

| Component | Component (wt %) |
|---|---|
| Sodium carbonate¹ | 15 |
| Sodium percarbonate² | 3 |
| C11 alcohol ethoxylate 7 E.O.³ | 3 |
| GDL⁴ | 5 |
| Citric acid⁵ | 59 |
| Sodium benzoate⁶ | 8 |
| Sodium Lauryl Sulfate⁷ | 7 |

¹= available from FMC Corporation, Philadelphia, PA, USA.
²= available from Solvay Chemicals, Galveston, TX, USA
³= available from Evonik Industries AG, Essen, Germany
⁴= available from Jungbunzlauer Suisse AG, Basel, Switzerland
⁵= available from S.A. Citrique Belge N.V., Tienen, Belgium
⁶= available from Emerald Kalama Chemical, Kalama, WA, USA
⁷= available from Stepan Company, Northfield, IL, USA

To prepare the effervescent solid dosage form composition, each of the indicated amounts of the components was placed in a laboratory scale vee-cone blender (Munson Machinery Company, Inc., Utica, NY). The components were blended for 5 minutes to achieve a homogeneous blend.

The homogeneous blend was used to prepare compressed tablets. A 5 gram aliquot of the homogeneous blend was weighed to be made into a compressed tablet. The 5 gram blend was compressed into a tablet using a 15/16″ round tool and die. Tablet compression was performed using a CARVER Press (Carver, Inc. (Wabash, IN)) and pressed at a gauge reading of 7 metric tons (7 tf (ton force-metric)). All of the tablets exhibited smooth face surfaces, had good edges and good sidewalls with few defects.

Aqueous solutions of the compositions were prepared as follows. The compressed tablet was dissolved in 700 mL of tap water (without stirring) at 19° C. The dissolution time for the tablet to completely dissolve was 12 minutes. The resulting solution had a pH of 4.7 and the solution was clear.

### Example 3—Foaming Hand Soap

| Component | Component (wt %) |
|---|---|
| Sodium carbonate¹ | 14 |
| GDL⁴ | 10 |
| Citric Acid⁵ | 52 |
| Sodium benzoate⁶ | 8 |
| Sodium Lauryl Sulfate⁷ | 9.5 |
| Sodium acetate⁸ | 4.5 |
| Fragrance | 2 |

¹= available from FMC Corporation, Philadelphia, PA, USA
⁴= available from Jungbunzlauer Suisse AG, Basel, Switzerland
⁵= available from S.A. Citrique Belge N.V., Tienen, Belgium
⁶= available from Emerald Kalama Chemical, Kalama, WA. USA
⁷= available from Stepan Company, Northfield, IL, USA
⁸= available from Niacet Corporation, Niagara Falls, NY, USA

A homogeneous blend was prepared as described in Examples 1 and 2.

The homogeneous blend was used to prepare compressed tablets. A 16 gram aliquot of the homogeneous blend was weighed to be made into a compressed tablet. The 16 gram blend was compressed into a tablet using a 1.25″ round tool and die. Tablet compression was performed using a CARVER Press (Carver, Inc. (Wabash, IN)) and pressed at a gauge reading of 7 metric tons (7 tf (ton force-metric)). All of the tablets exhibited smooth face surfaces, had good edges and good sidewalls with few defects.

Aqueous solutions of the compositions were prepared as follows. The compressed tablet was dissolved in 300 mL of tap water (without stirring) at 23° C. The dissolution time for the tablet to completely dissolve was 30 minutes. The resulting solution had a pH of 4.1 and the solution was clear.

### Example 4—Foaming Hand Soap

| Component | Component (wt %) |
|---|---|
| Sodium carbonate¹ | 28 |
| GDL⁴ | 14 |
| Citric Acid⁵ | 40 |
| Potassium sorbate⁹ | 8 |
| PEG 7 Glyceryl Cocoate¹⁰ | 2 |
| Alpha Olefin Sulfonate¹¹ | 7 |
| Fragrance | 1 |

¹= available from FMC Corporation, Philadelphia, PA, USA
⁴= available from Jungbunzlauer Suisse AG, Basel, Switzerland
⁵= available from S.A. Citrique Belge N.V., Tienen, Belgium
⁹= available from APAC Chem Corp., Nantong, China
¹⁰= available from Croda Inc., Mill Hall, PA
¹¹= available from Stepan Company, Northfield, IL

A homogeneous blend was prepared as described in Examples 1 and 2.

The homogeneous blend was used to prepare compressed tablets. A 9 gram aliquot of the homogeneous blend was weighed to be made into a compressed tablet. The 9 gram blend was compressed into a tablet using a 1.063″ round tool and die. Tablet compression was performed using a CARVER Press (Carver, Inc. (Wabash, IN)) and pressed at a gauge reading of 7 metric tons (7 tf (ton force-metric)). All of the tablets exhibited smooth face surfaces, had good edges and good sidewalls with few defects.

Aqueous solutions of the compositions were prepared as follows. The compressed tablet was dissolved in 300 mL of tap water (without stirring) at 22° C. The dissolution time for the tablet to completely dissolve was 25 minutes. The resulting solution had a pH of 5.6 and the solution was clear.

### Experimental Examples A Through D and Comparative Experimental Example 1

Exemplary formulations were prepared for preservative challenge testing using USP <51> Protocol testing. The United States Pharmacopeia (USP) is a globally recognized organization that specializes in testing medicine, foodstuffs and supplement products for safety and quality. One of the ways USP performs the task of ensuring product safety is to ensure that product preservation systems work to a specified standard. This specified standard for USP is the USP <51> Protocol.

The USP <51> Protocol challenges a chemical solution with five organisms. These challenge organisms are: A. brasiliensis, C. albicans, E. coli, P. aeruginosa, and S. aureus. The challenge test uses a microbial concentration of 1×10 5 CFU/mL. Each product to be tested has these organisms introduced in individual tests so the preservative can be evaluated by organism type. This preservative challenge was performed at room temperature and lasts at least for 28 days. The microbially challenged product with a preservative system was evaluated at day 14 and day 28. Test solutions were inoculated with the test microorganisms and plated on the appropriate sterile growth media agar plates for the type of organism used in the test. After incubation for the desired contact time, the incubated test substances were harvested and chemically neutralized. The number of microorganisms surviving after the conclusion of the testing period was determined by using established microbial serial dilution techniques for the agar plating of the microbes. A most-probable-number (MPN) was established for any surviving cells. Finally, a log reduction from the initial challenge concentration of each micro-organism was determined and checked against the pass/fail criteria of the USP <51> Protocol.

Products like hand soaps and hard surface cleaners are considered Category 2 products in the USP <51> Protocol. For these solutions, bacteria were evaluated separately from mold and yeast. The passing criteria for bacteria as a Category 2 product in the USP <51> Protocol is that the products must show at least a 2 log reduction by day 14 from the initial concentration of microbes introduced to the product with a preservative system, as well as no signs of further microbial growth from the 14 day readings. The passing criteria for mold and yeast require that no growth is seen at neither day 14 nor day 28 above a 0.5 log increase.

The following test organisms were used:
Aspergillus brasiliensis, ATCC 16404
Candida albicans, ATCC 10231
Escherichia coli, ATCC 8739
Pseudomonas aeruginosa, ATCC 9027
Staphylococcus aureus, ATCC 6538

The tested exemplary formulations for Comparative Experimental Example 1 and Experimental Examples A through D are shown in Table 1 below:

TABLE 1
Formulations.

| | Comparative | | | | |
|---|---|---|---|---|---|
| | Example 1 | Example A | Example B | Example C | Example D |
| | AllPurpose A | AllPurpose B | Degreaser | Glass Cleaner | Hand Soap |
| | % | % | % | % | % |
| Sodium carbonate¹ | 30 | 25 | 10 | 30 | 28 |
| Citric acid² | 38 | 48 | 35 | 60 | 42 |
| Sodium benzoate³ | 0.8 | 1 | 15 | 1 | 15 |
| GDL⁴ | 5.2 | 15 | 15 | 5 | 5 |
| Sodium acetate⁵ | 1 | 3 | 7 | 0 | 0 |
| Surfactant 1⁶ | 4 | 5 | 6 | 4 | 7 |
| C12-C18 | | | | | |
| ethoxylated alcohol | | | | | |
| Surfactant 2⁷ | 4 | 2 | 10 | 0 | 2 |
| Alkoxylated alcohol | | | | | |
| Fragrance | 2 | 1 | 2 | 0 | 1 |
| Sodium | 15 | 0 | 0 | 0 | 0 |
| percarbonate⁸ | | | | | |
| | 100 | 100 | 100 | 100 | 100 |
| Tablet weight (g) | 6 | 9 | 20 | 6 | 15 |
| Solvent for | | | | | |
| dissolution (g) | 294 | 291 | 480 | 294 | 285 |
| Final solution wt. (g) | 300 | 300 | 500 | 300 | 300 |
| Benzoate wt % in | 0.016 | 0.03 | 0.6 | 0.02 | 0.75 |
| solution | | | | | |
| Solution pH | 6.51 | 4.5 | 4.31 | 4.68 | 5.05 |

¹= available from FMC Corporation, Philadelphia, PA, USA
²= available from S.A. Citrique Belge N.V., Tienen, Belgium
³= available from Emerald Kalama Chemical, Kalama, WA. USA
⁴= available from Jungbunzlauer Suisse AG, Basel, Switzerland
⁵= available from Niacet Corporation, Niagara Falls, NY, USA
⁶= ECO Natrasense 125 (CAS No. 68213-23-0) available from Croda Inc., Mill Hall, PA
⁷= ECO Natrasense 265 (CAS No. 27252-75-1) available from Croda Inc., Mill Hall, PA
⁸= available from Solvay Chemicals, Galveston, TX, USA

The results of the preservative challenge testing using USP <51> Protocol testing are shown in Table 2 below.

TABLE 2
Preservative challenge testing results.

| | | log | | | log | | |
|---|---|---|---|---|---|---|---|
| | | reduction | log | log | reduction | log | |
| | | A. | reduction | reduction | P. | reduction | |
| Product | Day | brasiliensis | C. albicans | E. coli | aeruginosa | S. aureus | Pass/Fail |
| Comparative | 14 | 0.31 | >3.98 | 1.06 | >4.32 | >4.11 | fail |
| Example 1 | | | | | | | |
| Comparative | 28 | — | — | — | — | — | fail |
| Example 1 | | | | | | | |
| Example A | 14 | 0.11 | >3.98 | >4.29 | >4.32 | >4.11 | pass |
| Example A | 28 | 0 | >3.98 | 3.99 | >4.32 | >4.11 | pass |
| Example B | 14 | >3.98 | >3.98 | >4.29 | >4.32 | >4.11 | pass |
| Example B | 28 | >3.98 | >3.98 | 3.81 | >4.32 | >4.11 | pass |
| Example C | 14 | 0.23 | >3.98 | >4.29 | >4.32 | >4.11 | pass |
| Example C | 28 | 0 | >3.98 | >4.29 | >4.32 | >4.11 | pass |
| Example D | 14 | >3.98 | >3.98 | 2.93 | >4.32 | >4.11 | pass |
| Example D | 28 | >3.98 | >3.98 | >4.29 | >4.32 | >4.11 | pass |

As can be seen from the data above, Comparative Example 1, which had a solution pH greater than 6 (pH=6.51), with an amount of sodium benzoate in the final solution of 0.016 wt %, failed to meet the passing criteria of the preservative challenge testing per USP <51> Protocol. Experimental Examples A through D, which had a solution pH greater than 4 and less than 6, with an amount of sodium benzoate in the final solution in a range of 0.02 to 0.75 wt %, successfully met the passing criteria of the preservative challenge testing per USP <51> Protocol.

While various embodiments of the subject matter provided herein have been described, it should be understood that they have been presented by way of example only, and not limitation. Since modifications will be apparent to those of skill in this art, it is intended that this invention be limited only by the scope of the appended claims.

1. An effervescent solid dosage form composition, comprising:
a sugar acid lactone in an amount in the range of about 1 wt % to about 15 wt %, based on the total weight of the composition;
a preservative comprising salt of benzoic acid or sorbic acid or a combination thereof in an amount in the range of about 1 wt % to about 15 wt %, based on the total weight of the composition;
an acetate selected from among sodium acetate, potassium acetate, magnesium acetate, calcium acetate, and a combination thereof; and
an effervescence generator comprising:
(a) an acid in amount from about 20 wt % to about 60 wt % based on the total weight of the composition; and a base in an amount in the range of about 25 wt % to about 50 wt %, based on the total weight of the composition; or
(b) an acid in amount greater than 35 wt % but less than 60 wt % based on the total weight of the composition, and a base in an amount in the range of about 10 wt % to about 30 wt %, based on the total weight of the composition,
wherein:
the acid is a straight chain aliphatic carboxylic acid or a branched chain aliphatic carboxylic acid or a combination thereof; or
the acid is selected from the group consisting of acetic acid, adipic acid, citric acid, formic acid, fumaric acid, gluconic acid, glutaric acid, glycolic acid, lactic acid, maleic acid, malic acid, oxalic acid, succinic acid, glutaric acid, malonic acid, tartaric acid, and a combination thereof; or
the acid is selected from the group consisting of acetic acid, adipic acid, ascorbic acid, citric acid, formic acid, fumaric acid, gluconic acid, glutaric acid, glycolic acid, lactic acid, sorbic acid, succinic acid, sulfamic acid, and a combination thereof; and
the base comprises sodium bicarbonate, sodium carbonate, sodium sesquicarbonate, potassium carbonate, potassium bicarbonate, calcium carbonate, magnesium carbonate, magnesium oxide, sodium glycine carbonate, or a combination thereof.

2. (canceled)

3. The composition of claim 1, wherein the solid dosage form is an agglomerate, granule, tablet, pellet, puck, brick, briquette, block, layered compression, or composite.

4. The composition of claim 1, wherein sugar acid lactone is selected from the group consisting of allohepturonolactone, allonolactone, al luronol actone, altrohepturonolactone, altronolactone, altruronolactone, arabinolactone, arabinuronolactone, galactohepturonolactone, galactonolactone, galacturonolactone, glucohepturonolactone, gluconolactone, glucuronolactone, gulohepturonolactone, gulonolactone, guluronolactone, idohepturonolactone, idonolactone, iduronolactone, lyxuronolactone, mannohepturonolactone, mannonolactone, mannuronolactone, ribonolactone, riburonolactone, talohepturonolactone, talonolactone, taluronolactone, xylonolactone and xyluronolactone and a combination thereof; or
the sugar acid lactone is selected from the group consisting of a gluconolactone, a galactonolactone, a mannonolactone, a gulonolactone and a heptagluconolactone; or
the sugar acid lactone is a glucono-delta-lactone.

5.-6. (canceled)

7. The composition of claim 1, wherein the preservative comprises:
(a) sodium benzoate, potassium benzoate, calcium benzoate, or a combination thereof; or
(b) sodium sorbate, potassium sorbate, calcium sorbate, or any combination thereof;
or
(c) a combination of any one or more of (a) and any one or more of (b).

8. The composition of claim 1, further comprising a protective layer, wherein the protective layer comprises:
an acrylic polymer, a sugar, a starch, a maltodextrin, a polyethylene glycol, a film forming water soluble polymer, or a combination thereof; or
a coating material selected from among adipic acid, azelaic acid, glutaric acid, malonic acid, oxalic acid, pimelic acid, sebacic acid, suberic acid, succinic acid, undecanedioic acid, dodecanedioic acid, tridecanedioic acid, hydroxypropyl cellulose, hydroxypropyl methylcellulose, polyvinyl acetate, hydroxyethyl cellulose, methylhydroxyethyl cellulose, methyl cellulose, ethyl cellulose, cellulose acetate, sodium carboxymethyl cellulose, polymers and copolymers of acrylic acid and methacrylic acid and esters thereof, starch, modified starch, maltodextrin, a wax, gum arabic, shellac, water soluble polyvinyl alcohol, a polyalkylene glycol, sodium polyacrylate, polyvinylpyrrolidone, and combinations thereof.

9. (canceled)

10. The composition of claim 1, further comprising an additional component selected from among an organic solvent, a surfactant, a foaming agent, a buffering salt, a tablet lubricant, a fragrance, a colorant, a chelant, an enzyme, an acid, a carbonate, a bicarbonate, a percarbonate, a phosphate, sodium bisulfate, a wetting agent, a dispersing agent, a hydrotrope, a polyalkylene glycol, a polyglycol, a methoxypolyalkylene glycol, a polyglycol copolymer, a hexitol, a siloxane, a polysilane, a polysiloxane, sodium gluconate, a rheology control agent, a foam suppressant, a metal protectant, a corrosion inhibitor, a hydrogen peroxide generator in combination with a peracid catalyst, a bleaching agent, a bleach activator, an optical brightener, an anti-redeposition agent, and a combination thereof.

11. The composition of claim 10, wherein the surfactant is selected from among a cationic surfactant, an anionic surfactant, a non-ionic surfactant, a zwitterionic surfactant, a silicone surfactant, a biosurfactant, and a combination thereof, wherein the biosurfactant is a lecithin, a saponin, a rhamnolipid, a sophorolipid, a mannosylerythritol lipid, a marine alga glycolipid, a glucose lipid, a cellulose lipid, a trehalose lipid, an alkyl glucoside, a cellobiose lipid, a polyol lipid, a protein polyamine, a lipopolysaccharide, fengycin, iturin, lichenysin, surfactin, or a combination thereof.

12. The composition of claim 1, further comprising a humectant selected from among glycerin, diglycerin, triacetin, 1,3-butylene glycol, pentylene glycol, polyethylene glycol having a weight-average molecular weight of 800 or less, dipropylene glycol, panthenol, hyaluronic acid, sodium hyaluronate, and mixtures thereof, wherein the humectant is present in an amount of about 0.1 wt % to 10 wt % based on the weight of the solid dosage form.

13.-14. (canceled)

15. The composition of claim 1, further comprising an emollient selected from the group consisting of lanolin oil, cetyl alcohol, cetearyl alcohol, lauryl alcohol, oleyl alcohol, stearyl alcohol, PPG-15 stearyl ether, octyldodecanol, diisopropyl adipate, cetearyl octanoate. glycerin mono-, di-, and tri-esters, cholesterol, cholesterol esters, a linear silicone having a viscosity of from about 10 cP to about 100,000 cP at 25° C., a cyclomethicone, a light mineral oil, a petrolatum, a coconut oil, isopropyl myristate, soybean oil, stearic acid, caprylic acid, oleic acid, palmitic acid, lauric acid, octylpalmitate, octyl stearate, glyceryl stearate, and a combination thereof, wherein the emollient is present in an amount of about 0.1 wt % to 10 wt %, based on the weight of the solid dosage form.

16.-17. (canceled)

18. The composition of claim 1, further comprising a foam booster selected from among:
an aliphatic alcohol, an alkanolamide, and an amine oxide; or
PEG-75, PEG-100, monoethanolamide, diethanolamide, N-methylethanolamide, glycerylmonocaprylate, cocamidopropyl hydroxysultaine, sorbitan sesquicaprylate, dodecyl alcohol, alkanolamide, monoethanolamide, diethanolamide, N-methyl-ethanolamide, lauramidopropyl betaine, and cocoamidopropyl betaine,
wherein the foam booster is present in an amount of about 0.01 wt % to 15 wt %, based on the weight of the solid dosage form.

19.-22. (canceled)

23. A solution, comprising:
from about 50 wt % to about 99.95 wt % of a solvent; and
from about 0.05 wt % to about 50 wt % of the composition of claim 1 dissolved in the solvent,
wherein:
the solution has a pH from 4 to 6, and
the preservative of the composition is present in an amount from 0.02 to 1 wt % based on the weight of the solution.

24.-25. (canceled)

26. A method of disinfecting a surface, comprising:
dissolving a composition of claim 1 in a solvent to form a solution; and
applying the solution to the surface resulting in the destruction of, or prevention of the growth of, a microbe on the surface,
wherein:
the solvent used to dissolve the composition comprises water, an alcohol, an aldehyde, a ketone, or a combination thereof, and
the solution is applied to the surface by spraying, wiping, immersion or direct application or any combination thereof.

27.-28. (canceled)

29. A packaged system, comprising:
the effervescent solid dosage form composition of claim 1; and
a packaging material selected from the group consisting of glass, metal foil, treated metal foil, a metal foil pouch, plastic, plastic film, a plastic sheet, a blister pack, cardboard, a cardboard composite, paper and treated paper, and any combination thereof.

30. (canceled)

31. The packaged system of claim 29, further comprising a container for dissolving the composition in a solvent, or a receptacle for containing or dispensing the dissolved composition, or both.

32. The packaged system of claim 31, wherein the receptacle is selected from the group consisting of a spray bottle, a sponge, a conventional hand sprayer container, an electric spray dispenser container, a bucket, a can, a drum, a towelette, a wipe, a pad, and any combination thereof.

33. An article of manufacture, comprising:
the effervescent solid dosage form composition of claim 1; and
(a) a container suitable for containing the effervescent solid dosage form composition; or
(b) a set of instructions for preparing a cleaning solution or disinfectant solution by dissolving the effervescent solid dosage form composition in a solvent; or
(c) a set of instructions for storing the antimicrobial composition; or
(d) a material safety data sheet; or
(e) a dispenser or applicator for a solution prepared by dissolution of the effervescent solid dosage form composition; or
(f) any combination of two or more of (a), (b), (c), (d) and (e).

34. The article of manufacture of claim 33, comprising the container suitable for containing the effervescent solid dosage form composition, wherein the container comprises glass, acrylonitrile butadiene styrene (ABS), high impact polystyrene, polycarbonate, high density polyethylene, low density polyethylene, high density polypropylene, low density polypropylene, polyethylene terephthalate, polyethylene terephthalate glycol, polyvinylchloride, or a combination thereof.

35. The article of manufacture of claim 34, wherein the container further comprises a barrier film.
